(19) United States
(12) Patent Application Publication
(10) Pub. No.: US 2026/0249022 A1
(43) Pub. Date: Aug. 27, 2026

(54) MULTI-CARTRIDGE PUSH MODE DROPLET DELIVERY DEVICE

(71) Applicant: PNEUMA RESPIRATORY, INC., Boone, NC (US)

(72) Inventors: Charles Eric HUNTER, Boone, NC (US); Chao-Ping LEE, Shenzen City (CN); Jeffrey MILLER, Boone, NC (US); Chengjie LI, Shenzhen City (CN); Jianqiang LI, Shenzen City (CN); Shi-Bo WANG, Shenzen City (CN); Matthew CULPEPPER, Boone, NC (US); Caley MODLIN, Boone, NC (US); Cristian SALAZAR, Boone, NC (US); Madison JOHNSON, Boone, NC (US); Michael SCOGGIN, Boone, NC (US)

(21) Appl. No.: 19/547,512

(22) Filed: Feb. 23, 2026

Related U.S. Application Data
(60) Provisional application No. 63/761,703, filed on Feb. 21, 2025.
(60) Provisional application No. 63/767,935, filed on Mar. 6, 2025.
(60) Provisional application No. 63/774,453, filed on Mar. 19, 2025.
(60) Provisional application No. 63/800,862, filed on May 6, 2025.
(60) Provisional application No. 63/891,593, filed on Oct. 1, 2025.
(60) Provisional application No. 63/899,515, filed on Oct. 15, 2025.
(60) Provisional application No. 63/904,941, filed on Oct. 24, 2025.

(51) Int. Cl. A61M 11/00; A61M 15/00

(52) U.S. Cl. CPC A61M 11/005; A61M 11/003; A61M 15/0003; A61M 15/0021; A61M 15/0085; A61M 2202/0007; A61M 2202/049; A61M 2205/0294; A61M 2210/1039

(57) ABSTRACT

An electronic droplet delivery device with an inhalation mouthpiece that includes multiple fluid reservoirs and multiple aperture plates having one or more transducers with a membrane or multiple membranes between the one or more transducers and the aperture plates. Multiple fluid reservoirs allow for different fluids, such as medicines, nicotine, chemicals for marijuana, and flavorings to be separately provided from separate reservoirs into a resulting combined droplet stream inhaled by a user through the mouthpiece. In embodiments, the multiple reservoirs, multiple aperture plates and one or more transducers can all be contained within a housing of the droplet delivery device, including an in-line configuration within a linear housing.

### CROSS-REFERENCE TO RELATED APPLICATIONS

This application claims the benefit of priority of U.S. Provisional App. No. 63/761,703, filed Feb. 21, 2025, U.S. Provisional App. No. 63/767,935, filed Mar. 6, 2025, U.S. Provisional App. No. 63/774,453, filed March 19,2025, U.S. Provisional App. No. 63/800,862, filed May 6, 2025, U.S. Provisional App. No. 63/891,593, filed Oct. 1, 2025, U.S. Provisional App. No. 63/899,515, filed Oct. 15, 2025, and U.S. Provisional App. No. 63/904,941, filed Oct. 24, 2025, all of which are incorporated herein by reference in their entireties.

### FIELD OF THE INVENTION

The present disclosure relates generally to aerosol and droplet delivery systems for inhalation and, more particularly, to electronic droplet delivery devices using one or more non-heated ejection systems (e.g., piezoelectric push-mode and/or ring-mode ejectors) to generate and deliver one or more streams of droplets from one or more fluid reservoirs, including devices having multiple cartridges and multiple ejector plates configured to deliver different droplet size distributions to different regions of a user's respiratory system.

### BACKGROUND OF THE INVENTION

Inhalable delivery systems are used to deliver active agents and other constituents to the respiratory tract. In some applications (e.g., nicotine replacement, consumer nicotine products, and respiratory therapeutics), it can be desirable to deliver different formulations and/or different dose amounts during an inhalation.

Conventional heated electronic nicotine delivery systems (ENDS) and other vaporization devices can generate thermal decomposition products and may provide limited control over droplet/particle size and regional deposition. In addition, some ingredients (e.g., nicotine salts, acids, certain sensates, flavors, or therapeutics) can be irritating or undesirable when deposited in certain regions of the respiratory tract.

Further, some formulations are difficult to combine in a single reservoir because they may be immiscible, unstable when mixed, or require different solvents or excipients. Separate reservoirs and ejection systems can also provide redundancy, allow different dosing regimens, and enable tailoring of droplet size and plume geometry to target the mouth/throat, upper airway, and/or lower airway/lungs.

The following patents and patent applications are incorporated herein by reference in their entirety: U.S. Pat. No. 9,962,507, U.S. Pat. No. 9,956,360, U.S. Patent Application Publication No. 2022/022658, U.S. Pat. No. 11,793,945, U.S. Pat. No. 12,161,795, U.S. Patent Application Publication No. 2021/0236745, U.S. Patent Application Publication No. 2023/0286008, U.S. Patent Application Publication No. 2023/0356253, International Published Application Nos. WO 2023/064477, WO 2025/101933, WO 2023/172677, WO 2023/215389, WO 2024/020046, WO 2024/020041, WO 2024/020047, WO 2024/151663, WO 2025/006626, and International Application Nos. PCT/US2024/031401, PCT/US2024/58487, PCT/US2025/013898, PCT/US2025/018391, PCT/US2025/018389, PCT/US2025/024305, PCT/US2025/024579, and PCT/2025/049918.

### SUMMARY OF THE INVENTION

In embodiments, the present disclosure provides a droplet delivery device including a housing having a mouthpiece at an airflow exit, multiple fluid reservoirs (e.g., removable cartridges and the like), and one or more ejection assemblies configured to eject one or more streams of droplets into an airflow for inhalation by a user.

In some embodiments, the device includes a plurality of ejector plates each having a plurality of apertures extending through the thickness of the plate. A piezoelectric vibrating member (e.g., a piezoelectric disc bonded to a horn) is operably coupled to one or more of the ejector plates, with a membrane positioned between the vibrating member and the ejector plate(s) such that ultrasonic energy is transferred to the ejector plate(s) to eject droplets from the apertures.

In some embodiments, a first reservoir supplies a first formulation (e.g., a nicotine-containing formulation) to a first ejector plate configured to generate a first droplet size distribution having a high respirable fraction suitable for lower airway/lung delivery, and a second reservoir supplies a second formulation (e.g., a flavor and/or sensate formulation) to a second ejector plate configured to generate a second droplet size distribution that preferentially deposits in the mouth and/or upper airway.

In some embodiments, the device includes a barrier and/or separate flow paths within the mouthpiece to reduce mixing of the streams. In some embodiments, the device is configured such that ejection from different ejector plates is time-multiplexed and/or modulated (e.g., by pulse width modulation, duty cycle control, and/or power control) to control dosing and reduce interaction between droplets from different streams.

In some embodiments, an ejector plate configured for lower airway delivery is positioned rearward of an ejector plate configured for upper airway delivery, optionally defining an open volume or increased path length to promote evaporation and/or size reduction of droplets prior to exiting the mouthpiece.

In some embodiments, the device includes two or more vibrating members respectively coupled to two or more ejector plates. In other embodiments, a single vibrating member is configured to drive two or more ejector plates (e.g., via multiple protrusions and/or one or more coupling rods/extensions) to reduce part count.

In further embodiments, the device includes electronics (e.g., one or more printed circuit board assemblies, batteries, sensors, and user interface components) configured to detect a user inhalation and/or user input and to control actuation parameters for each ejector plate, including actuation timing, duration, duty cycle, and power level.

These and other features and advantages of the present disclosure will be apparent from the following detailed description and the accompanying drawings.

### BRIEF DESCRIPTION OF THE DRAWINGS

FIGS. 1A and 1B illustrate two embodiments of a multi-cartridge device including one ejector plate behind another. The lower airway ejector plate (114) delivers an ejected stream of droplets to the lungs. The lower airway ejector plate (114) is behind the upper airway ejector plate (113) that delivers an ejected stream of droplets to the upper airway. The lower airway ejector plate (114) is positioned parallel to the upper airway ejector plate (113). In the embodiment shown in FIG. 1A, the ejected stream of droplets from the lower airway ejector plate flows (114) around the upper airway ejector plate (113) in front of it via one pathway. In the embodiment shown in FIG. 1B, this stream of droplets flows around the upper airway ejector plate (113) via two or more pathways. In these embodiments, the ejector plates are centered along a central axis. Additionally, there may or may not be a barrier (112) between the two or more ejected streams of droplets.

FIG. 2 illustrates an embodiment where the ejector plate that delivers an ejected stream of droplets to the lungs is to the rear of the ejector that delivers an ejected stream of droplets to the upper airways. The ejector plate meant to deliver to the lungs is positioned perpendicular to the ejector plate meant to deliver to the upper airways. There may or may not be a barrier between the two ejected streams of droplets.

FIG. 3 illustrates an embodiment where the ejector plate meant to deliver an ejected stream of droplets to the lungs is behind the ejector plate meant to deliver an ejected stream of droplets to the upper airways. The ejector plates are not centered along a central axis, and therefore they are off-center. There may or may not be a barrier between the two ejected streams of droplets.

FIG. 4 illustrates an embodiment where the ejector plate meant to deliver an ejected stream of droplets to the lungs is positioned much further behind the ejector plate meant to deliver an ejected stream of droplets to the upper airways. The lower airway aerosol 12 travels through an open volume 411 that assists in the evaporation of the droplets. The droplets that are ejected evaporate very quickly, and with an additional path length of the open volume, the droplets will evaporate. The open volume may also increase in cross sectional area to decrease the velocity of the droplets and increase the time for the droplets to evaporate.

FIGS. 5A and 5B illustrate an embodiment where a vibrating member for one of the ejector plates is located behind the other vibrating member. The vibrating members can be aligned along a central axis, a common axis (not necessarily central) or in other alignments. The vibrating member in the back is coupled with an associated ejector plate to transfer vibrations to the ejector plate. This arrangement can be accomplished with a rod or extension coupled to the vibrating member. A barrier may or may not be arranged between the two ejected streams of droplets.

FIGS. 6A and 6B and FIGS. 7A and 7B illustrate embodiments where one vibrating member is used to create an ejected stream of droplets for each ejector plate. As nonlimiting examples, FIGS. 6A and 6B illustrate an example where the angle between both ejector plates is less than 180 degrees. As another nonlimiting examples, FIGS. 7A and 7B illustrate an example where the angle between both ejector plates is greater than 180 degrees.

FIGS. 8A and 8B illustrates another example of using one vibrating member to create an ejected stream of droplets for each ejector plate. In this embodiment, the vibrating member is coupled to the secondary ejector plate with a rod or an extension to transfer vibrations to that ejector plate.

FIGS. 9A and 9B illustrate another example of using one vibrating member to create an ejected stream of droplets for multiple ejector plates. In this embodiment, a vibrating member with multiple extrusions is used. FIGS. 9A and 9B show a nonlimiting example of a vibrating member with multiple extrusions.

FIG. 10 illustrates a cross-sectional computer aided design drawing (CAD) of a droplet delivery device with two ejectors. This design includes a mouthpiece, two cartridges, two vibrating members, two ejector plates, an exit port, two airway paths, a handpiece, a button, two batteries, and two printed circuit board assemblies (PCBAs).

FIGS. 11A and 11B illustrate external CAD images of the same device shown in FIG. 10. The mouthpiece and cartridges are removable (shown coupled in FIG. 11A) as FIG. 11B shows the device without the mouthpiece and cartridges.

FIG. 12A and 12B illustrates CAD images of the cartridge and mouthpiece of an embodiment. FIG. 12A shows a cross-sectional view of the cartridges and mouthpiece with the airflow exit and both aerosol pathways.

FIGS. 13A and 13B illustrate CAD images of a device with two ejectors in a box-like design. The ejector brackets are located below the cartridges. This non-limiting example can include one or two batteries. The design can have one or two vibrating members, which are located below the ejector brackets.

FIGS. 14A-C illustrates CAD images of external views of a droplet delivery device with four ejectors and four cartridges.

FIG. 15 illustrates an exploded view of the device of FIGS. 14A-C that has four ejectors and four cartridges.

FIGS. 16A and 16B illustrate cross-sectional views of the device shown in FIGS. 14A-C and FIG. 15.

### DETAILED DESCRIPTION

### Multiple Reservoir Push Mode Droplet Device

Definitions. As used herein, the term “aerosol” refers to a suspension of droplets and/or particles in a gas flow (e.g., air) for inhalation. The term “droplets” includes liquid droplets and droplets that partially or fully evaporate prior to exiting the mouthpiece. The term “fluid” includes solutions, mixtures, emulsions, suspensions, and dispersions.

As used herein, “respirable fraction” refers to the proportion of emitted aerosol droplets and/or particles having an aerodynamic diameter within a size range suitable for lower airway delivery (e.g., less than about 5 μm). Unless otherwise specified, respirable fraction may be determined using any recognized aerosol particle sizing methodology, including but not limited to cascade impaction, inertial impaction, laser diffraction, or aerodynamic particle sizing, under controlled laboratory conditions at a defined airflow rate representative of device operation. The particular measurement technique, airflow rate, and environmental conditions may vary depending on the testing protocol employed, and values reported herein are intended to encompass measurements obtained using pharmaceutically and scientifically accepted particle sizing methods.

As used herein, the terms “approximately,” “about,” and “substantially” refer to typical manufacturing and measurement tolerances (e.g., ±10% unless context indicates otherwise). Ranges may be combined and sub-ranges are expressly contemplated. Unless otherwise indicated, stated ranges include endpoints.

Reference numerals in the drawings correspond to like features across embodiments. Directional terms such as “front,” “rear,” “upper,” “lower,” “left,” and “right” are used for convenience of description with respect to the orientation shown in the figures and do not limit the claims.

There is a need for the ability to deliver two or more fluids to the respiratory system. One need is to deliver the fluids to two or more different areas in the respiratory system. In this case, the droplet size of the aerosol is key to delivering fluid to a specific area of the respiratory system. For example, it is generally accepted that droplets smaller than about 4 μm are delivered to the alveolar region of the lungs.

Another need is to deliver two or more fluids that are immiscible. The two or more fluids could be delivered to the same area of the respiratory system or different areas of the respiratory system.

Yet another need is to deliver two or more fluids to the respiratory system. This could be because the fluids require different dosing regimens. This could be to deliver a larger quantity of fluid in one dose. Additionally, it could be that the active agent is present in a low percentage of the overall fluid and more fluid is required to deliver the required dose, such as those from non-soluble or sparingly soluble components or agents.

The present disclosure provides a droplet delivery device for delivery of one or more fluid(s) as an ejected stream of droplets to the respiratory system of a user. Not intending to be limiting, some embodiments of the droplet delivery device include a push-mode type of delivery system or a ring-mode type of delivery system. Additionally, the droplet delivery device could include both push-mode and ring-mode types of delivery systems. The droplet delivery device may use any type of ejection system suitable for creating a stream of droplets from fluid.

In other embodiments, the droplet delivery device includes a heated type of delivery system such as a heating coil, ceramic heater, or other inductive, conductive, or convective methods of heating. In some embodiments the droplet delivery device may include one or more heating elements to deliver one or more fluids. In some embodiments the droplet delivery device may include one or more heating elements to deliver one or more fluids, in addition to one or more alternative delivery systems, such as a push-mode or ring-mode delivery system.

In another embodiment, the droplet delivery device includes a heating system that heats the liquid to change one or more of the liquid's characteristics to enhance the ejection of the liquid. As a non-limiting example, as the liquid increases in temperature the surface tension decreases.

In another embodiment, the droplet delivery device includes a heating system that heats the liquid to ensure that it stays above a minimum temperature. In a non-limiting example, this can be used to ensure the liquid does not freeze.

The droplet delivery device delivers an ejected stream of droplets through an ejector plate with a plurality of apertures through its thickness. In one embodiment, each fluid has its own ejector plate. In another embodiment, two or more fluids can be ejected from a singular ejector plate. Additionally, another embodiment can have a combination of the two, where one or more fluids are delivered to one or more ejector plates.

In some embodiments, push-mode technology is used for aerosol generation. Push mode uses a vibrating member (115), which may comprise a piezoelectric disc bonded to a horn, in conjunction with an ejector plate (113, 114) which has apertures formed through its thickness. The vibrating member (115) and ejector plate (113, 114) are separated by a membrane (116). The vibrating member (115) transfers ultrasonic energy to the ejector plate (113, 114) to generate aerosol without the use of heat. The ejector plate is held in an ejector bracket (1017), which is connected to a removable cartridge (1012) that holds the liquid to be aerosolized.

In some embodiments, the vibrating member is driven at an ultrasonic frequency (e.g., about 20 kHz to about 300 kHz, or about 50 kHz to about 200 kHz) and at a power level selected to achieve a desired ejected mass and droplet size distribution. Drive parameters may include frequency, voltage amplitude, burst length, duty cycle, and waveform shape. In some embodiments, the device includes a driver circuit configured to actively tune frequency (e.g., to a resonance of the vibrating member and/or ejector assembly) based on sensed current/voltage phase, impedance, temperature, and/or emitted aerosol feedback.

In some embodiments, the ejector plate comprises a metal (e.g., stainless steel, nickel, titanium), ceramic, polymer, or semiconductor substrate having a thickness of about 10 μm to about 500 μm. Apertures may be formed by laser drilling, electroforming, photochemical etching, micro-machining, or other suitable techniques, and may be cylindrical, tapered, or multi-stage to control capillary pressure and droplet detachment. In some embodiments, the apertures are arranged in an array and include an inlet side and an outlet side, optionally with hydrophilic and/or hydrophobic surface treatments (e.g., coatings) to control priming and leakage.

In some embodiments, the membrane between the vibrating member and ejector plate comprises a polymer film (e.g., polyimide), metal foil, elastomer, or composite diaphragm, and is configured to transmit vibration while providing a liquid seal. The membrane may be bonded, clamped, welded, over-molded, or otherwise secured to the ejector plate and/or a supporting frame, and may include one or more gaskets or compliant features to maintain sealing under tolerance stack-up.

In some embodiments, each cartridge includes a reservoir body, a fill port and/or septum, one or more vents, and a fluid outlet configured to deliver fluid to the ejector plate. Fluid communication may be provided via a capillary channel, wick, microfluidic conduit, valve, or porous element. In some embodiments, a check valve and/or burstable seal prevents leakage during shipping and is opened upon cartridge insertion. In some embodiments, cartridge insertion provides both a fluidic connection (e.g., via a pierceable seal) and an electrical connection (e.g., via contacts) to power and control the associated ejector assembly.

In some embodiments, the housing and mouthpiece define one or more airflow paths that entrain the emitted droplets. The device may include one or more air inlets, and the airflow exit may be shaped to produce a desired plume (e.g., divergent, convergent, or coaxial). In some embodiments, the barrier and/or separate channels maintain separation between at least two emitted streams until after exiting the mouthpiece. In some embodiments, the barrier extends at least partially along the length of the mouthpiece and may include one or more baffles or turns to promote deposition of larger droplets in the mouth region while allowing smaller droplets to pass toward the lower airway.

In some embodiments, an open volume (e.g., a plenum) is provided downstream of one or more ejector plates to increase residence time, reduce velocity, and/or promote evaporation of droplets. The open volume may expand in cross-sectional area and may include features that reduce impaction (e.g., smooth radii) while maintaining low pressure drop.

In some embodiments, the device includes one or more sensors including an inhalation sensor (e.g., pressure sensor, flow sensor, microphone, and/or thermal anemometer), a temperature sensor, a cartridge presence sensor, and/or a liquid level sensor. The controller may initiate actuation in response to detected inhalation and may terminate actuation based on a timer, an inhalation profile, and/or a dose limit. In embodiments, dose limits may be enforced per puff, per unit time, and/or per day.

In an embodiment, there are two ejector plates in a droplet delivery device. One of the ejector plates delivers a fluid that is meant to be delivered deep into the respiratory system, or lungs. The other ejector plate delivers a fluid that is meant to be delivered in the upper respiratory system or upper airways, such as the mouth or throat. This is done for several reasons: delivering a flavor to the mouth and lungs to make the inhalation pleasant, ensuring only the necessary fluid is delivered to the lungs, and not delivering unnecessary substances to the lungs.

In another embodiment, there are more than one ejector plates. Each of the ejector plates delivers a different agent to the lungs. This eliminates the need to combine the agents into one solution. Each ejector plate can be configured to deliver the fluid at different volumes or times. In a non-limiting example, one fluid may be delivered at 5 mg per inhalation and another can be delivered at 3 mg per inhalation (there can be more than two ejector plates). Each ejector plate can be configured to deliver fluid between 0.1 mg to 100 mg. Additionally, each ejector plate can be configured to deliver at different times or frequencies. In a non-limiting example, one ejector plate is configured to deliver fluid 3 times a day and the other ejector plate is configured to deliver fluid 1 time a day. The ejector plates can be configured to deliver fluid between 0 to 10,000 times a day.

In another embodiment, a greater volume of fluid is required for delivery than what is delivered with one ejector plate. With more than one ejector plate, additional fluid can be delivered to the respiratory system. This means the more than one ejector plates all deliver the same fluid.

In another embodiment, the greater volume of fluid being delivered to the lungs is used to deliver agents that are insoluble or sparingly soluble in water or other solvents. In a non-limiting example, if the fluid can only have a maximum of 1% of a particular agent, additional ejector plates will double, triple, quadruple the amount of the agent delivered.

In some embodiments, an ejector plate meant to deliver a fluid to the lungs can have an average aperture diameter of between about 1 μm to 3 μm. Aperture diameters this small create droplets that are delivered deeper in the respiratory system. Most of these droplets will not impact the mouth or upper airways. This ejector plate can have a respirable fraction of equal to or greater than 90% under defined test conditions (e.g., as measured by an impactor and/or laser diffraction at a specified flow rate), indicating that a high proportion of the emitted droplets/particles are within a size range suitable for lower airway/lung delivery.

In an embodiment, an ejector plate meant to deliver a fluid to the lungs delivers a solution containing nicotine. Nicotine formulations can be irritating and/or harsh and can cause an unpleasant sensory experience if deposited in the mouth or upper airways; therefore, in some embodiments it may be desirable to minimize deposition of the nicotine-containing solution in the upper airway of the respiratory system.

In another embodiment, all of the more than one ejector plates deliver nicotine to the respiratory system.

In some embodiments, an ejector plate meant to deliver a fluid to the mouth and/or throat can have an average aperture diameter of between about 2 μm to 15 μm. This ejector plate is designed to deliver the ejected stream of droplets in the upper respiratory system, meaning that most of the aerosol is deposited in the mouth and/or upper airways. A preferred embodiment of this ejector plate produces aerosol with a respirable fraction of equal to or less than 20%.

In an embodiment, an ejector plate meant to deliver a fluid to the mouth and/or throat delivers a solution containing a flavor. The flavor is intended to provide the user with a pleasant sensory experience and taste.

In one embodiment, one or more of the ejectors deliver a numbing agent or a sensate to distract or numb the targeted area before during and or after another substance contacts the targeted area. In non-limiting examples, a caustic substance is delivered to the targeted area. In another non-limiting example, larger droplets are delivered to the targeted area. In some embodiments, a droplet delivery device contains an ejector plate meant to deliver fluid to the lungs and a second ejector plate meant to deliver fluid to the mouth and/or throat. In some embodiments, the two ejectors are supplied with different fluids; the ejector plate meant to deliver fluid to the lungs delivers a nicotine-containing solution, and the ejector plate meant to deliver fluid to the mouth and/or throat delivers a solution containing a flavor.

In some embodiments, the ejector plate meant to deliver aerosol to the lungs can have an average aperture diameter of between about 1 μm to 2 μm, about 1 μm to 3 μm, about 1 μm to 4 μm, about 1 μm to 5 μm, about 1 μm to 10 μm, about 1 μm to 25 μm, or less than 1 μm. The respirable fraction of the aerosol created by the ejector can be greater than 50%, greater than 60%, greater than 70%, greater than 80%, or greater than 90%.

In some embodiments, the ejector plate meant to deliver aerosol to the mouth and/or throat can have an average aperture diameter of between about 2 μm and about 30 μm (e.g., about 2 μm to about 15 μm, about 5 μm to about 25 μm, or about 10 μm to about 30 μm), selected to preferentially deposit aerosol in the mouth and/or upper airway. In some embodiments, the emitted aerosol from such an ejector plate has a respirable fraction of less than about 50% (e.g., less than about 40%, less than about 30%, less than about 20%, or less than about 10%), under defined test conditions.

The ejector plate meant to deliver aerosol to the upper airways can be used with pulse width modulation (PWM). The ejection can be adjusted to increase or decrease the amount of upper airway droplets delivered to the user. The PWM can be anywhere between 0% and 100%. As a nonlimiting example, the upper airway droplets can be delivered 45% of the time of inhalation. This can be done with PWM where the ejector plate turns on and off. As another nonlimiting example, the ejector plate can be on for 45 ms and off for 55 ms to deliver 45% of the time. This can be set during manufacturing, or the user can change the amount.

The use of PWM is possible due to the fact that the ejector plates can be rapidly turned on and off with low latency to initiate droplet ejection. By contrast, many heated aerosol generation systems exhibit a warm-up time on the order of tenths of a second before reaching a steady output, which can limit fine time-domain modulation of emitted mass. Accordingly, in some embodiments the disclosed non-heated ejection technology enables rapid modulation of output during an inhalation to adjust dose and/or sensory experience.

In other embodiments, any or all of the ejector plates can be controlled with PWM regardless of their targeted area(s) in the respiratory system.

In some embodiments, a droplet delivery device may have 3, 4, or 5 ejector plates. In some embodiments a droplet delivery device may have more than 5 ejector plates. In some embodiments, the 3 or more ejector plates may all be in fluid communication with a single fluid-containing reservoir or with multiple fluid-containing reservoirs. In other embodiments, the 3 or more ejector plates may be in fluid communication with 3 or more fluid-containing reservoirs. In other embodiments, 2 or more of the ejector plates may be in fluid communication with 1 fluid-containing reservoir, and 1 or more of the ejector plates may be in fluid communication with a different fluid-containing reservoir. In any embodiment, the fluid containing reservoir(s) may contain the same fluid/formulation or different fluids/formulations.

As a non-limiting example, a droplet delivery device may have 3 or more ejector plates, 2 or more of which are in fluid communication with a nicotine-containing solution and 1 or more of which is in fluid communication with a flavor-containing solution. The ejector plates in fluid communication with a nicotine-containing solution may be designed to eject solution at droplet sizes that will be delivered to the lower respiratory tract. The ejector plate(s) in fluid communication with a flavor-containing solution may be designed to eject solution at droplet sizes that will deposit in the mouth and/or throat. Having 2 or more ejectors deliver nicotine-containing solution may allow the use of solution(s) with a lower concentration of nicotine, as compared to having only one ejector. Users may be able to receive the same, or higher, dose of nicotine with less irritation.

As a non-limiting example, a droplet delivery device may have 3 or more ejector plates, all of which are in fluid communication with a nicotine-containing solution. In some embodiments, the nicotine-containing solution may also have a flavor component.

As a non-limiting example, a droplet delivery device may have 3 or more ejector plates, 1 or more of which are in fluid communication with a nicotine-containing solution and 2 or more of which are in fluid communication with a flavor-containing solution. The ejector plate(s) in fluid communication with a nicotine-containing solution may be designed to eject solution at droplet sizes that will be delivered to the lower respiratory tract. The ejector plates in fluid communication with a flavor-containing solution may be designed to eject solution at droplet sizes that will deposit in the mouth and/or throat.

In one embodiment, a droplet delivery device has 4 ejector plates designed to eject solution at small droplet sizes (e.g., about 1 μm, less than 1 μm, less than 0.8 μm, less than 0.6 μm, less than 0.4 μm, etc.) deliverable to the lower respiratory tract. The 4 ejector plates are in fluid communication with a solution containing nicotine at a concentration between 0.2% (w/w) and 1.8% (w/w) (e.g., 0.2% (w/w), 0.3% (w/w), 0.4% (w/w), 0.45% (w/w), 0.5% (w/W), 0.6% (w/w), 0.8% (w/w), 1.0% (w/w), 1.2% (w/w), 1.4% (w/w), 1.6% (w/w), 1.8% (w/w), etc.). The solution may contain other excipients, and the nicotine may be in the form of a nicotine salt. The ejector plates may be in fluid communication with 4 separate fluid reservoirs or may be in fluid communication with one fluid reservoir.

In one embodiment, a droplet delivery device has 3 ejector plates designed to eject solution at small droplet sizes (e.g., about 1 μm, less than 1 μm, less than 0.8 μm, less than 0.6 μm, less than 0.4 μm, etc.) deliverable to the lower respiratory tract and 1 ejector plate designed to eject solution at droplet sizes deliverable to the mouth and/or throat. The 3 ejector plates designed to deliver small droplet sizes are in fluid communication with a solution containing nicotine at a concentration between 0.2% (w/w) and 1.8% (w/w) (e.g., 0.2% (w/w), 0.3% (w/w), 0.4% (w/w), 0.45% (w/w), 0.5% (w/w), 0.6% (w/w), 0.8% (w/w), 1.0% (w/w), 1.2% (w/w), 1.4% (w/w), 1.6% (w/w), 1.8% (w/w), etc.). The solution may contain other excipients, and the nicotine may be in the form of a nicotine salt. The 3 ejector plates may be in fluid communication with 3 separate fluid reservoirs or may be in fluid communication with one fluid reservoir. The ejector plate designed to deliver solution to the mouth and/or throat is in fluid communication with a flavor containing solution.

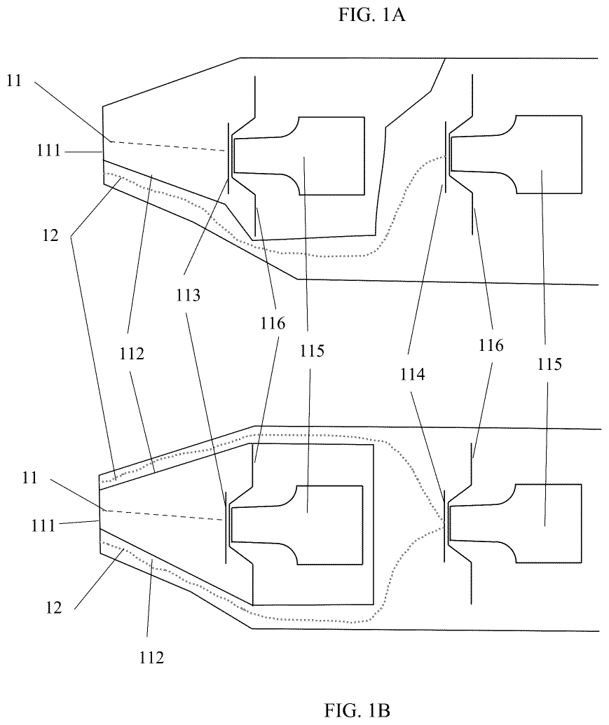

As a non-limiting example, FIGS. 1A and 1B illustrate two embodiments of a two or more cartridge device design where one ejector plate is behind the other. One ejector plate is meant to deliver an ejected stream of droplets to the droplets to the lungs, the lower airway ejector plate (114), and is behind an ejector plate meant to deliver an ejected stream of droplets to the upper airways, the upper airway ejector plate (113). The lower airway ejector plate is positioned parallel to the upper airway ejector plate.

In the embodiment shown in FIG. 1A, the lower airway aerosol (12), flows around the upper airway ejector plate (113) via one pathway. In the embodiment shown in FIG. 1B, the lower airway aerosol (12), flows around the upper airway ejector plate (113) via two or more pathways. The upper airway ejector plate generates upper airway aerosol (11). Both aerosols exit through the airflow exit (111). In both embodiments shown in FIG. 1, the ejector plates are centered along a central axis. Additionally, there may or may not be a barrier (112) between the two or more ejected streams of droplets.

The embodiments shown in FIGS. 1A and 1B illustrate designs using push-mode technology, which uses a vibrating member (115) and ejector plate (113, 114), separated by a membrane (116), to create aerosol with ultrasonic energy without the use of heat. In the embodiments in FIG. 1, each ejector plate is paired with its own vibrating member (115).

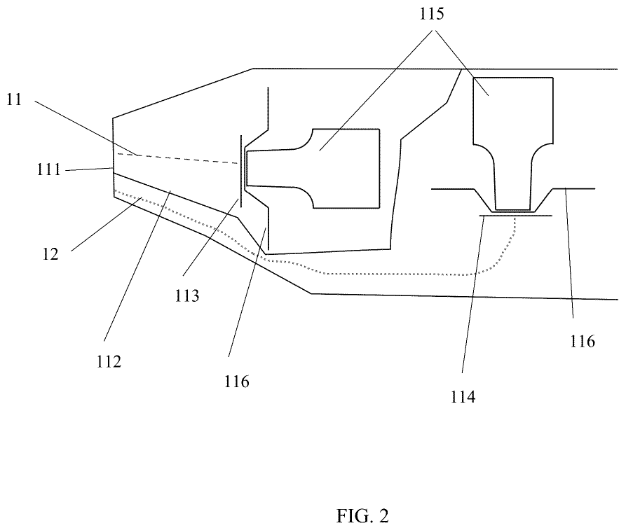

As a non-limiting example, FIG. 2 illustrates an embodiment where a lower airway ejector plate (114) is behind an upper airway ejector plate (113). The lower airway ejector plate (114) is positioned perpendicular to the upper airway ejector plate (113). There may or may not be a barrier (112) between the upper airway aerosol (11) and the lower airway aerosol (12). The aerosols exit through the airflow exit (111) for inhalation. The embodiment shown in FIG. 2 illustrates push-mode technology, with each ejector plate paired to its own vibrating member (115), separated by membranes (116).

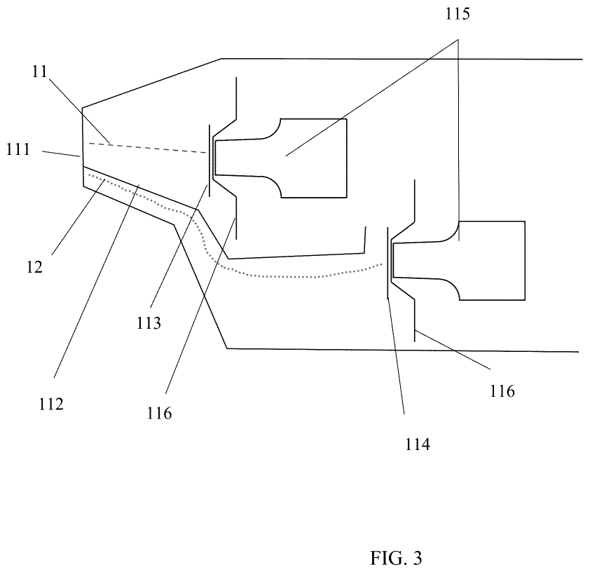

As a non-limiting example, FIG. 3 illustrates an embodiment where a lower airway ejector plate (114) is behind an upper airway ejector plate (113). The ejector plates are not centered along a central axis, and therefore they are off-center. There may or may not be a barrier (112) between the upper airway aerosol (11) and the lower airway aerosol (12). The aerosols exit through the airflow exit (111) for inhalation. The embodiment shown in FIG. 3 illustrates push-mode technology, with each ejector plate paired to its own vibrating member (115), separated by membranes (116).

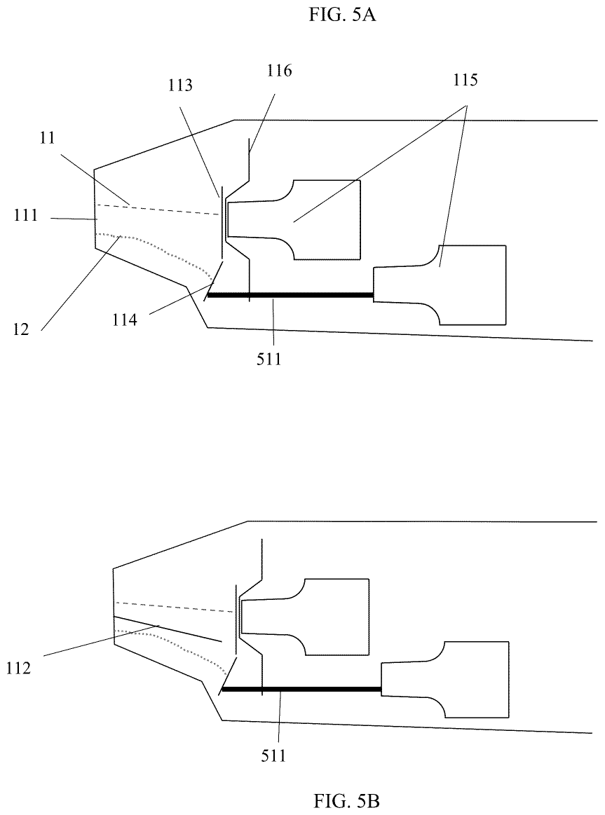

As a non-limiting example, FIGS. 5A and 5B illustrate embodiments with push-mode technology where a vibrating member (115) for one ejector plate is located behind another vibrating member (115). The vibrating members (115) can be aligned along a central axis or can be offset from one another. The vibrating member (115) in the back is coupled with an associated ejector plate to transfer vibrations to the ejector plate. This can be accomplished with a rod (511) or extension coupled to the vibrating member (115). In the embodiments shown in FIGS. 5A and 5B, a lower airway ejector plate (114) is adjacent to an upper airway ejector plate (113). Both ejector plates are paired to vibrating members (115), which are separated from the ejector plates by a membrane (116). The lower airway ejector plate (114) is paired to a vibrating member (115) via a rod (511). In the embodiment shown in FIG. 5A, there is no barrier between the upper airway aerosol (11) and the lower airway aerosol (12). In the embodiment shown in FIG. 5B, there is a barrier (112) between the two aerosols. In both embodiments, the aerosols exit through the airflow exit (111) for inhalation.

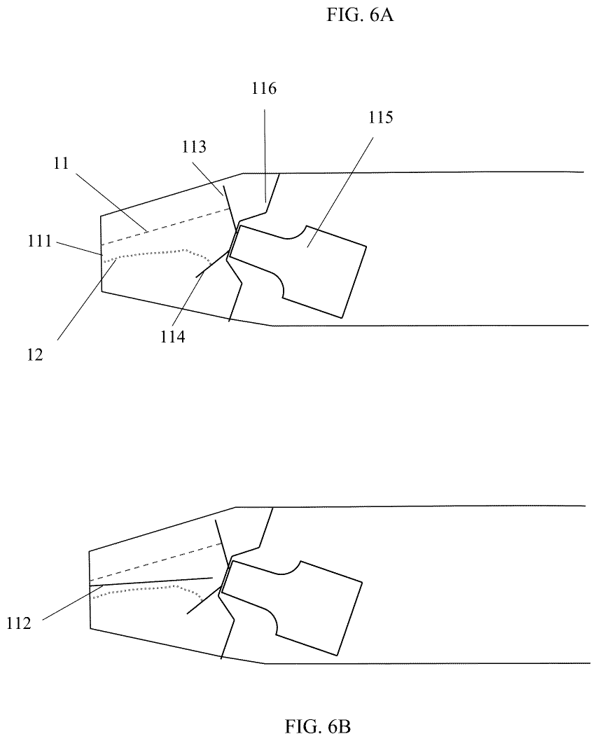

FIGS. 6A and 6B and FIGS. 7A and 7B illustrate embodiments using push-mode technology where one vibrating member (115) is used to create an ejected stream of droplets from multiple ejector plates. As a nonlimiting example, FIG. 6 illustrates an example where the angle between two ejector plates (113, 114) is less than 180 degrees. Both ejector plates are paired to a single vibrating member (115), which is separated by a membrane (116). The embodiment shown in FIG. 6A does not have a barrier (112) between the two aerosols (11, 12). The embodiment shown in FIG. 6B has a barrier (112) between the two aerosols (11,12). In both embodiments, the aerosols exit through the airflow exit (111) for inhalation.

As another nonlimiting example, FIGS. 7A and 7B illustrate an example where the angle between two ejector plates (113, 114) is greater than 180 degrees. Both ejector plates are paired to a single vibrating member (115), which is separated by a membrane (116). The embodiment shown in FIG. 7A does not have a barrier (112) between the two aerosols (11, 12). The embodiment shown in FIG. 7B has a barrier (112) between the two aerosols (11,12). In both embodiments, the aerosols exit through the airflow exit (111) for inhalation.

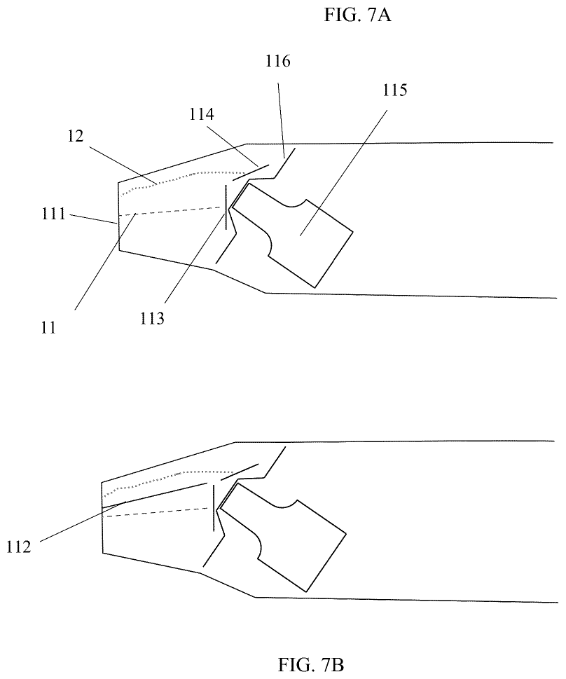

As a non-limiting example, FIGS. 8A and 8B illustrate embodiments with push-mode technology, using one vibrating member (115) to create an ejected stream of droplets for multiple ejector plates. In the embodiments shown in FIGS. 8A and 8B, the vibrating member (115) is behind one ejector plate (114), separated by a membrane (116), and is also paired to a secondary ejector plate (113) via a rod (511) or an extension to transfer vibrations to that ejector plate. In this way, the vibrating member (115) can transfer vibrations to both ejector plates (113, 114). The embodiment shown in FIG. 8A does not have a barrier (112) between the two aerosols (11, 12). The embodiment shown in FIG. 8B has a barrier (112) between the two aerosols (11,12). In both embodiments, the aerosols exit through the airflow exit (111) for inhalation.

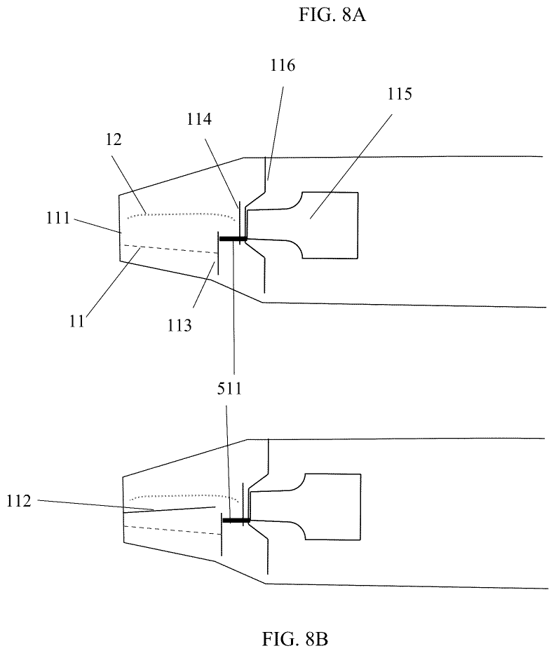

As a non-limiting example, FIGS. 9A and 9B illustrate another embodiment of using one vibrating member to create an ejected stream of droplets for multiple ejector plates. In this embodiment, a vibrating member with multiple extrusions (911) is used. FIGS. 9A and 9B show a nonlimiting example of a vibrating member with multiple extrusions (911). In the embodiment shown in FIGS. 9A and 9B each extrusion on the vibrating member is paired to an ejector plate (113, 114). In this way, the vibrating member can transfer vibrations to both ejector plates (113, 114). The ejector plates are separated from the vibrating member by a membrane (116). The embodiment shown in FIG. 9A does not have a barrier (112) between the two aerosols (11, 12). The embodiment shown in FIG. 9B has a barrier (112) between the two aerosols (11,12). In both embodiments, the aerosols exit through the airflow exit (111) for inhalation.

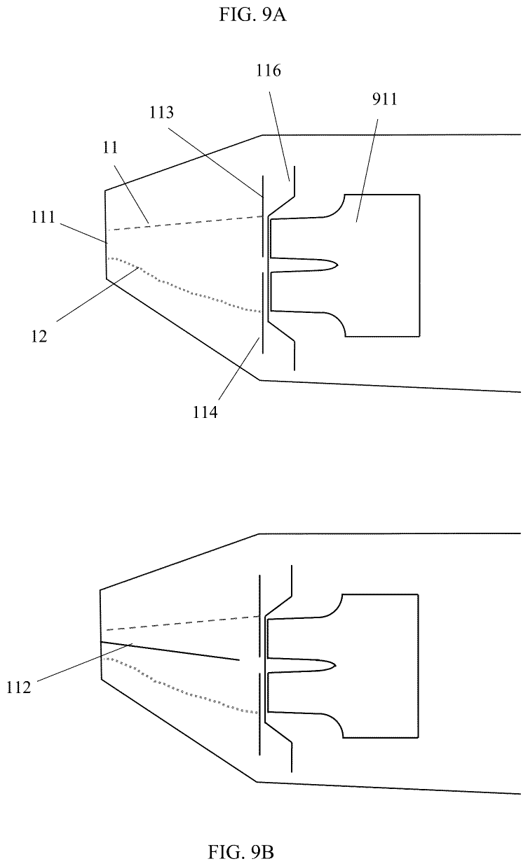

As a non-limiting example, FIG. 10 illustrates a cross sectional computer aided design (CAD) of a push-mode device embodiment with two ejectors. This embodiment includes two ejector plates (113, 114), which are each paired with a vibrating member (115). The vibrating members (115) are separated from the ejector plates (113, 114) by membranes (116). The ejector plates (113, 114) are each held in separate ejector brackets (1017). Each ejector is paired to a removable cartridge (1012). A mouthpiece (1011) encompasses both cartridges (1012). The handpiece (1013) includes two batteries (1015) and two printed circuit board assemblies (PCBAs, 1016). A button (1014) is included on the handpiece (1013) for user interfacing. The embodiment shown in FIG. 10 includes a barrier (112) between the two aerosol streams, which exit through the airflow exit (111) for inhalation.

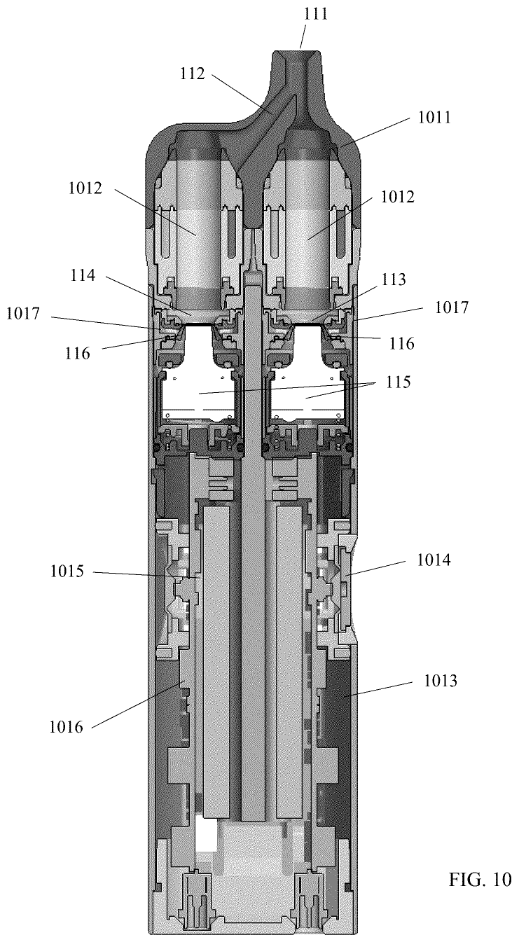

FIGS. 11A and 11B illustrate an external CAD image of the same device shown in FIG. 10. The mouthpiece (1011) and cartridges (1012) are removable. The ejector plates (113, 114) are each held in separate ejector brackets (1017), which are below the cartridges (1012). FIG. 11B shows the device without the mouthpiece (1011) and cartridges (1012). The handpiece (1013) has a button (1014) and LED lights (1018) for user interfacing.

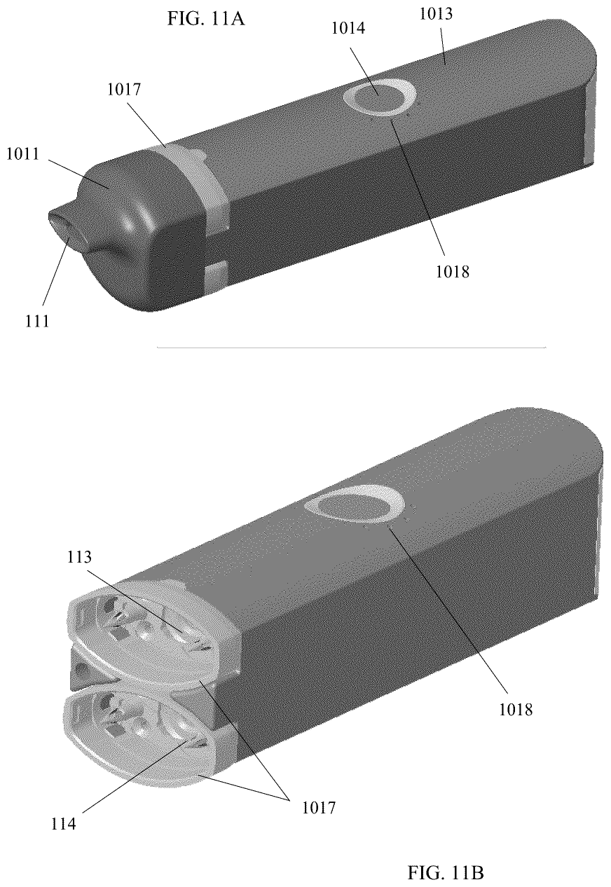

As a non-limiting example, FIGS. 12A and 12B illustrate CAD images of the cartridges (1012) and mouthpiece (1011) of an embodiment that includes two fluid reservoirs. FIG. 12A shows a cross section of the cartridges (1012) and mouthpiece (1011) with the airflow exit (111) and both aerosol pathways. The embodiment shown in FIGS. 12A and 12B includes a barrier (112) between the two aerosol pathways. FIG. 12B is an external view of the embodiment.

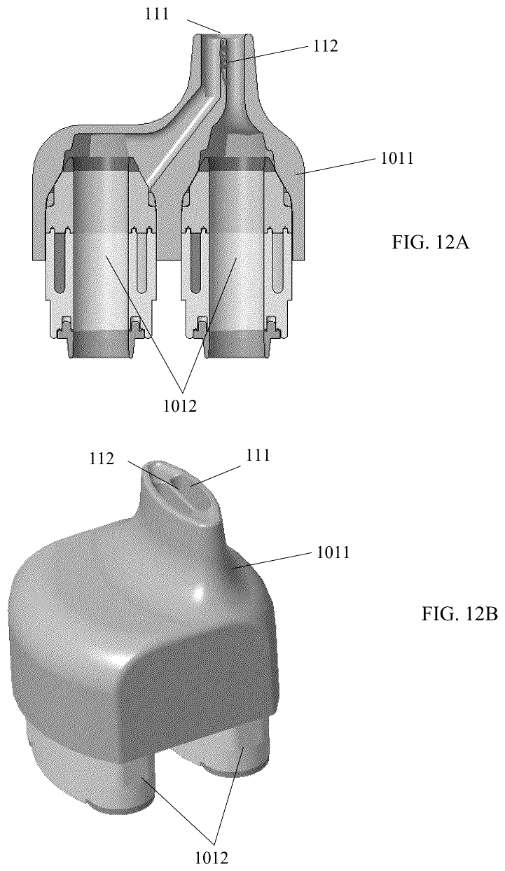

As a non-limiting example, FIG. 13 illustrates CAD images of a device with two ejectors and two cartridges (1012) in a more box-like design. The two ejector plates (113, 114) are each held in separate ejector brackets (1017) and are located below the cartridges (1012). This design can have one or two batteries (1015). The design can have one or two vibrating members (115), which are located below the ejector brackets (1017). The embodiment shown in FIG. 13 features two PCBAs (1016) and one battery (1015). The handpiece (1013) has a button (1014) for user interfacing.

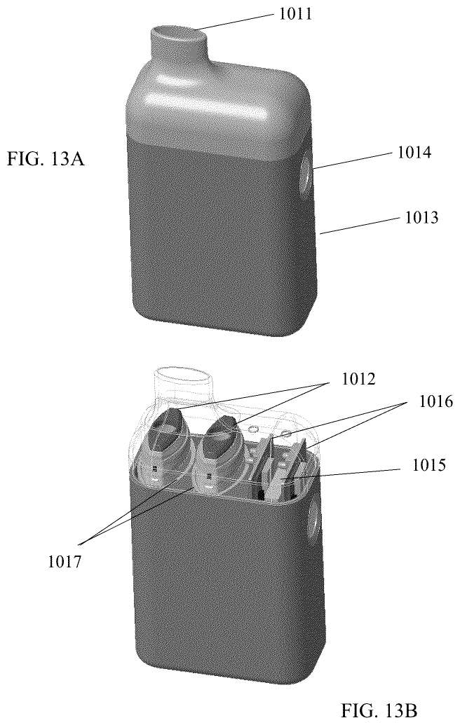

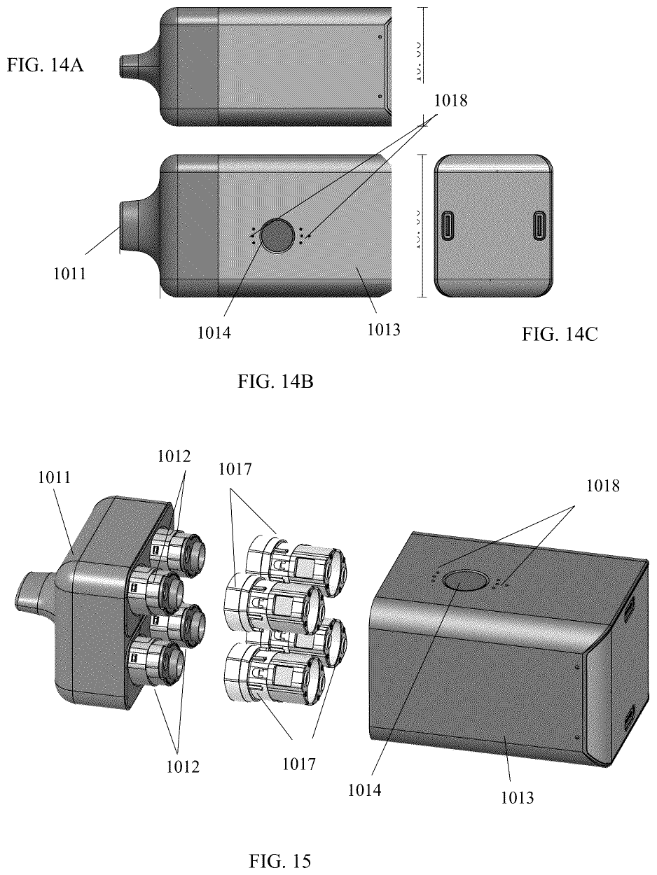

As a non-limiting example, FIGS. 14A-C illustrates CAD images of a push-mode device with four ejectors and four cartridges. A button (1014) and LED lights (1018) are included on the handpiece (1013) for user interfacing. FIG. 15 shows an exploded view of the device in FIG. 14. The four cartridges (1012) are each paired to a separate ejector bracket (1017). A mouthpiece (1011) encompasses the cartridges (1012). The mouthpiece (1011) and cartridges (1012) are removable.

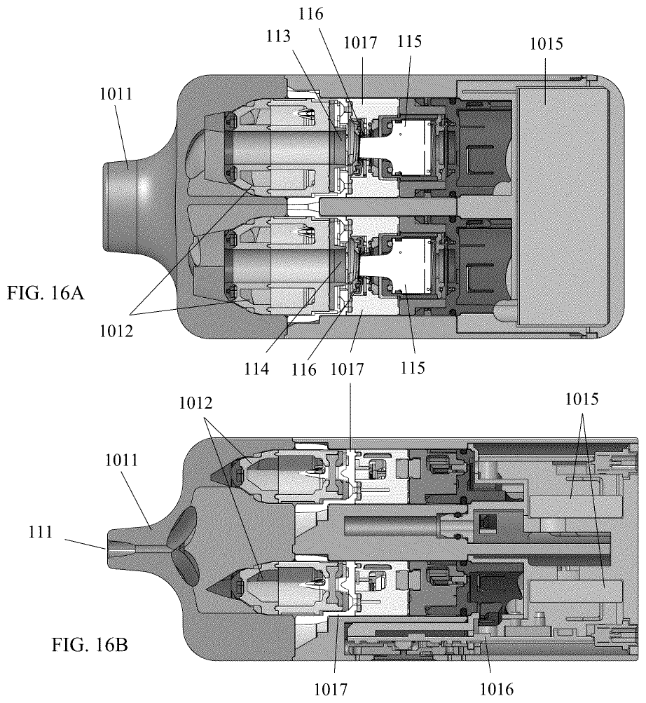

Further to the non-limiting example, FIGS. 16A and 16B shows two cross sectional views of the device in FIGS. 14A-C and FIG. 15. FIG. 16A is a top view cross section, and FIG. 16B is a side view cross section. A mouthpiece (1011) encompasses the four cartridges (1012). The four cartridges (1012) are each paired to a separate ejector bracket (1017). The ejector brackets (1017) each hold an ejector plate (113, 114) and are each paired to a vibrating member (115). The vibrating members (115) are separated from the ejector plates (113, 114) by membranes (116). The device can include one or more batteries (1015) and one or more PCBAs (1016). One PCBA (1016) and two batteries (1015) are shown in FIG. 16. All four aerosols exit through the airflow exit (111) for inhalation. There may or may not be a barrier between the aerosols.

In some embodiments, a higher percentage of nicotine may be used with an ejector plate designed to create small droplets. This may be used in a device that also has a second ejector plate designed to deliver to the mouth and/or throat supplied with a flavor-containing solution. As a non-limiting example, a device may have an ejector plate supplied with a solution containing nicotine at a concentration of 5-20% (w/w) designed to deliver droplets to the lower respiratory tract and a second ejector plate supplied with a flavor-containing solution designed to deliver droplets to the mouth and/or throat. The ejector plate supplied with the nicotine-containing solution may be designed to deliver small droplets (e.g., about 1 μm, less than 1 μm, less than 0.8 μm, less than 0.6 μm, less than 0.4 μm, etc.).

Supplying a solution containing a high concentration of nicotine to an ejector plate designed to deliver smaller droplets may allow for a user to receive a satisfying amount of nicotine with less irritation.

As a non-limiting example, a device may have two or more ejector plates supplied with a solution containing nicotine at a concentration of 5-20% (w/w) designed to deliver droplets to the lower respiratory tract and one or more ejector plates supplied with a flavor-containing solution designed to deliver droplets to the mouth and/or throat. The ejector plate supplied with the nicotine-containing solution may be designed to deliver small droplets (e.g., about 1 μm, less than 1 μm, less than 0.8 μm, less than 0.6 μm, less than 0.4 μm, etc.).

### Open Volume

In one embodiment, an ejector plate meant to deliver a stream of droplets to the lungs and/or lower airways is positioned further back than an ejector plate meant to deliver aerosol to the upper airways, relative to the mouthpiece (1011). This increase in distance increases the amount of travel time for the droplets created by the ejector meant to deliver aerosol to the lungs and/or lower airways, thereby increasing the amount of evaporation of the droplets. This could lead to the droplets being smaller when they exit the mouthpiece (1011). The open volume could also have a wider cross sectional area to decrease the velocity of the droplets, increasing the amount of evaporation of the droplets.

In another embodiment, any or all of the ejector plates will have an increased distance and or open volume pathway before the airflow exit regardless of their targeted area(s) in the respiratory system.

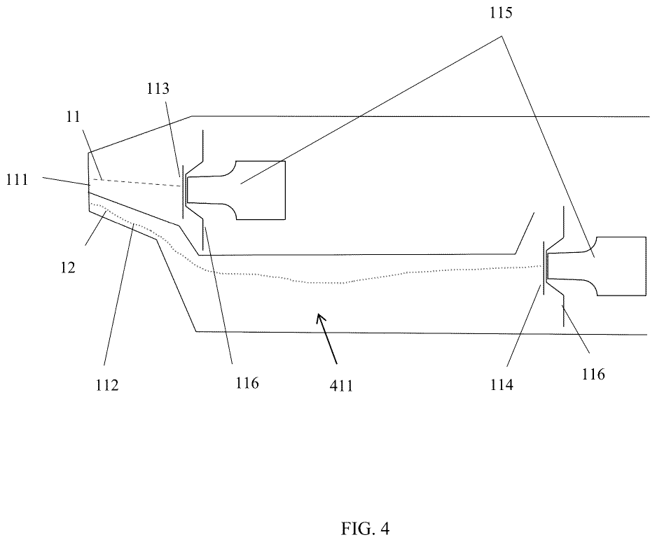

As a nonlimiting example, FIG. 4 illustrates an embodiment where a lower airway ejector plate (114) is positioned much further behind an upper airway ejector plate (113). The lower airway aerosol (12) travels through an open volume (411) meant to assist in the evaporation of the droplets. In FIG. 4, the ejector plates are not centered along a central axis, and therefore they are off-center. There may or may not be a barrier (112) between the upper airway aerosol (11) and the lower airway aerosol (12). The aerosols exit through the airflow exit (111) for inhalation. The embodiment shown in FIG. 4 illustrates push-mode technology, with each ejector plate paired to its own vibrating member (115), separated by membranes (116).

### Fluids

In some embodiments, one fluid with one formulation can be provided to one liquid reservoir which supplies all ejector plates in a device.

In some embodiments, two or more fluids can be used in the device. The two or more fluids can contain the same formulation or different formulations. The two or more formulations can be provided to two or more liquid reservoirs which supply ejector plates independently. Various embodiments of fluids, also called compositions, are listed below.

In some embodiments, compositions herein are composed of water at a concentration of about 80% (w/w) to about 95% (w/w). In some embodiments, the compositions herein may comprise about 60% (w/w) to about 80% (w/w) water. In some embodiments, the compositions herein may comprise about 80% (w/w) to about 100% (w/w) water. In some embodiments, the compositions herein may comprise about 0% (w/w) to about 60% (w/w) water.

In some embodiments, compositions herein comprise nicotine at a concentration of 0% (w/w) to 5% (w/w). In some embodiments, compositions herein comprise nicotine in a concentration of greater than 5% (w/w). In some embodiments, the composition does not include any nicotine in the formulation.

In some embodiments, compositions herein may comprise L-lactic acid at a concentration of 0% (w/w) to about 5% (w/w). In some embodiments, compositions herein may comprise L-lactic acid at a concentration of about 5% (w/w) to about 20% (w/w). In some embodiments, compositions herein may comprise L-lactic acid at a concentration greater than 20% (w/w). In some embodiments, the composition does not include any L-lactic acid.

In some embodiments, compositions herein may comprise of propylene glycol at a concentration of 0% (w/w) to about 20% (w/w). In some embodiments, compositions herein may comprise of propylene glycol at a concentration of about 20% (w/w) to about 100% (w/w). In some embodiments, the composition does not include any propylene glycol in the formulation.

In some embodiments, compositions herein may comprise ethanol at a concentration of 0% (w/w) to about 10% (w/w). In some embodiments, compositions herein may comprise ethanol at a concentration of 10% (w/w) to about 100% (w/w). In some embodiments, the composition does not include any ethanol in the formulation.

In some embodiments, compositions herein may comprise L-menthol. The concentration of L-menthol in embodiments can range from about 0.1% (w/w) to about 1.0% (w/w). In other embodiments, the concentration of L-menthol can be higher than 1.0%. In order to create a soluble solution with L-menthol in water, ethanol and propylene glycol are needed. For a water and/or water-nicotine based solution, propylene glycol needs to be between 5% (w/w) to about 15% (w/w) and ethanol needs to be between 1% (w/w) and about 10% (w/w). For an L-menthol solution at a concentration of 0.8% (w/w) or lower, the minimum amount of ethanol is 2.5% (w/w), and the minimum amount of propylene glycol is 10% (w/w). For L-menthol solutions at a concentration of above 0.8% (w/w), ethanol needs to be between 2.5% (w/w) to 10% (w/w) with 10% (w/w) propylene glycol.

In some embodiments, compositions herein may comprise WS-23 (2-isopropyl-N,2,3-trimethylbutyramide) in the concentration of 0.1% (w/w) to about 0.5% (w/w). In other embodiments, formulations may include WS-23 in a concentration higher than 0.5% (w/w). In order to use WS-23 in a water based formulation, ethanol concentration of 2.5% (w/w) to about 10% (w/w) may be needed. In addition, propylene glycol at a concentration of 10% (w/w) may also be needed.

In some embodiments, compositions herein may comprise of concentrated fruit extracts in the concentration of 0.1% (w/w) to about 5% (w/w). In some embodiments, compositions herein may comprise of concentrated fruit extracts in the concentration of about 5% (w/w) to about 10% (w/w). In some embodiments, compositions herein may comprise of concentrated fruit extracts in concentrations greater than 10% (w/w).

In another embodiment, compositions herein comprised of concentrated fruit extract could require additional excipients in order to get the fruit extract into water-based solutions. Ethanol in the concentration of 1% (w/w) to 10% (w/w) might be needed. Ethanol may be used in a concentration greater than 10% (w/w) as needed.

In another embodiment, compositions herein comprised of concentrated fruit extract could require additional excipients in order to get the fruit extract into water-based solutions. Propylene glycol in the concentration of 1% (w/w) to about 15% (w/w) might be needed. Propylene glycol may be used in a concentration greater than 10% (w/w) as needed.

In another embodiment, compositions herein comprised of concentrated fruit extract could require additional excipients in order to get the fruit extract into water-based solutions. Ethanol in the concentration of 1% (w/w) to 10% (w/w), in addition to propylene glycol in the concentration of 1% (w/w) to about 15% (w/w), might be needed. In some embodiments, ethanol in the concentration of 1% (w/w) to 10% (w/w) may be used without propylene glycol to get the fruit extract into water-based solutions. In some embodiments, propylene glycol in the concentration of 1% (w/w) to about 15% (w/w) may be used without ethanol to get the fruit extract into water-based solutions.

In some embodiments, compositions herein may comprise of cyclodextrin at a concentration of 0.1% (w/w) to about 5% (w/w). In some embodiments, compositions herein may comprise of cyclodextrin at a concentration greater than 5% (w/w). Cyclodextrin is used as a solubilizing agent.

In another embodiment, cyclodextrin can be used by itself, or it can be used with ethanol at a concentration of 1% (w/w) to about 10% (w/w). In other embodiments, cyclodextrin can be used with ethanol at a concentration greater than 10% (w/w).

In another embodiment, cyclodextrin can be used with propylene glycol in a concentration of 1% (w/w) to about 15% (w/w). In other embodiments, cyclodextrin can be used with propylene glycol at a concentration greater than 15% (w/w).

In another embodiment, cyclodextrin can be used with both ethanol and propylene glycol.

In some embodiments, compositions herein comprise of flavoring chemicals in the concentration of 0.001% (w/w) to about 5% (w/w). In some embodiments, compositions may comprise flavoring chemicals at concentrations of greater than 5% (w/w).

In another embodiment, flavoring chemicals can be used by themselves, or can be used with ethanol at a concentration of 1% (w/w) to about 10% (w/w). In other embodiments, flavoring chemicals can be used with ethanol at a concentration greater than 10% (w/w).

In another embodiment, flavoring chemicals can be used with propylene glycol in a concentration of 1% (w/w) to about 15% (w/w). In other embodiments, flavoring chemicals can be used with propylene glycol at a concentration greater than 15% (w/w).

In another embodiment, flavoring chemicals can be used with cyclodextrin in a concentration of 1% (w/w) to about 5% (w/w). In other embodiments, flavoring chemicals can be used with cyclodextrin in a concentration greater than 5% (w/w).

In another embodiment, flavoring chemicals can be used with a combination of ethanol, propylene glycol, and/or cyclodextrin.

In one embodiment, a fluid containing nicotine and/or flavor can be supplied to one ejector, and a cannabinoid solution can be supplied to a second ejector.

In one embodiment, a solution may comprise cannabinoid(s), including but not limited to THC and/or CBD, at a concentration of 0.1% (w/w) to about 30% (w/w). That solution can contain 40% (w/w) to about 60% (w/w) ethanol, 5% to about 10% (w/w) glycerol, 5% (w/W) to about 25% (w/w) propylene glycol, and/or 5% (w/w) to about 50% (w/w) water.

In other embodiments, cannabinoid(s) can be in a solution with excipients at any concentrations to create a fluid that can be supplied to an ejector plate to create aerosol. The excipients can include, but are not limited to ethanol, glycerol, propylene glycol, and/or water.

In some embodiments, a solution to deliver flavor can be used with active pharmaceutical ingredient (API) drugs. A device can include a liquid reservoir, or cartridge (1012), containing a solution with flavor and a second cartridge (1012) containing a solution with one or more APIs such as, but not limited to, albuterol sulfate, levalbuterol, formoterol, salmeterol, indacaterol, ipratropium bromide, tobramycin, tiotropium, glycopyrronium, aclidinium, fluticasone, budesonide, mometasone, or a combination of them. The liquid reservoirs can each be in fluid communication with a separate ejector plate.

In some embodiments, a solution to deliver flavor can be used with biologics drugs. A device can include a liquid reservoir, or cartridge (1012), containing a solution with flavor and a second cartridge (1012) containing a solution with one or more biologics drugs such as, but not limited to, therapeutic peptides, proteins, antibodies, and other bioengineered molecules include: growth factors, insulin, vaccines (Prevnor—Pneumonia, Gardasil—HPV), antibodies (Avastin, Humira, Remicade, Herceptin), Fc Fusion Proteins (Enbrel, Orencia), hormones (Elonva—long acting FSH, Growth Hormone), enzymes (Pulmozyme—rHu-DNAase-), other proteins (Clotting factors, Interleukins, Albumin), gene therapy and RNAi, cell therapy (Provenge—Prostate cancer vaccine), antibody drug conjugates-Adcetris (Brentuximab vedotin for HL), cytokines, anti-infective agents, polynucleotides, oligonucleotides (e.g., gene vectors), or any combination thereof; or solid particles or suspensions such as Flonase (fluticasone propionate) or Advair (fluticasone propionate and salmeterol xinafoate). The liquid reservoirs can each be in fluid communication with a separate ejector plate.

In some embodiments, two or more different biologics can be used in the same device with one biologic in each liquid reservoir. Biologics drugs could include, but are not limited to, therapeutic peptides, proteins, antibodies, and other bioengineered molecules include: growth factors, insulin, vaccines (Prevnor—Pneumonia, Gardasil—HPV), antibodies (Avastin, Humira, Remicade, Herceptin), Fc Fusion Proteins (Enbrel, Orencia), hormones (Elonva—long acting FSH, Growth Hormone), enzymes (Pulmozyme—rHu-DNAase-), other proteins (Clotting factors, Interleukins, Albumin), gene therapy and RNAi, cell therapy (Provenge—Prostate cancer vaccine), antibody drug conjugates—Adcetris (Brentuximab vedotin for HL), cytokines, anti-infective agents, polynucleotides, oligonucleotides (e.g., gene vectors), or any combination thereof; or solid particles or suspensions such as Flonase (fluticasone propionate) or Advair (fluticasone propionate and salmeterol xinafoate). The liquid reservoirs can each be in fluid communication with a separate ejector plate. Alternatively, the liquid reservoirs could each be in fluid communication with the same ejector plate configured to eject the fluids at different times.

In some embodiments, two or more different APIs can be used in the same device with one API in each liquid reservoir. APIs can include, but are not limited to, albuterol sulfate, levalbuterol, formoterol, salmeterol, indacaterol, ipratropium bromide, tobramycin, tiotropium, glycopyrronium, aclidinium, fluticasone, budesonide, mometasone, or a combination of them. The liquid reservoirs can each be in fluid communication with a separate ejector plate. Alternatively, the liquid reservoirs could each be in fluid communication with the same ejector plate configured to eject the fluids at different times.

In some embodiments, each liquid reservoir is in fluid communication with an ejection system that can deliver APIs and/or biologics at different size ranges. In some embodiments, the particle size of the aerosol produced can range from about 0.7 μm to about 3 μm. In some embodiments, the particle size of the aerosol produced can range from about 3 μm to about 6 μm. In some embodiments, the particle size of the aerosol produced can range from about 0.5 μm to about 2 μm. In some embodiments, the particle size of the aerosol produced can range from about 2 μm to about 4 μm. In some embodiments, the particle size of the aerosol produced can range from about 4 μm to about 7 μm.

In some embodiments, one of the fluids is a nicotine solution. This solution is meant to be delivered to the lungs and/or lower airways for rapid uptake. This solution can be a simple nicotine salt solution and can have minimal ingredients. In some embodiments, the nicotine formulation will include water, nicotine, and an acid. The acid can be lactic acid, benzoic acid, hydrochloric acid, or the like. The nicotine solution can be supplied to an ejector plate meant to deliver an ejected stream of droplets to the lungs and/or lower airways.

Additionally, in a preferred embodiment, one of the fluids is a solution containing a flavor. The flavor formulation can have food grade ingredients. The flavor solution can be supplied to an ejector plate meant to deliver a stream of droplets to the upper airways. The flavor is meant to deposit in the mouth and throat.

In some embodiments, a device is configured with a liquid reservoir containing a nicotine-containing solution in fluid communication with an ejector plate meant to deliver aerosol to the lower airways and a second liquid reservoir containing a flavor solution in fluid communication with a second ejector plate meant to deliver aerosol to the upper airways.

In some embodiments, the concentration of nicotine in a solution influences the delivered droplet size in a water-based solution. As the nicotine concentration is reduced, the particle size is also reduced. The reduction of the particle size is attributed to the evaporation of the water within the particle.

Minimizing the amount of ingredients delivered to the lungs has the potential to make the device less harmful to use than other products used to inhale nicotine.

Minimizing the amount of ingredients involved in the formulation, whether it is a nicotine solution or flavor solution, results in a higher surface tension. This higher surface tension results in increased ejected mass for a given ejector plate aperture diameter driven at a given power level. This makes it easier to deliver desirable levels of nicotine to users. A higher surface tension also makes it easier to prevent liquid from seeping through the apertures in the ejector plate. A lower surface tension liquid will seep through the apertures easier.

In another embodiment, the nicotine formulation can include a flavor component in the formulation. Smaller droplets can deliver the additional flavor to other areas of the respiratory system. It could be impactful to have a cooling component in the nicotine solution to reduce any irritation experienced from inhaling the nicotine stream of droplets.

In some embodiments, two or more nicotine-containing formulations are supplied to two or more ejector plates. The nicotine-containing formulations can be the same or can be different from each other. They may contain different concentrations of nicotine, different concentrations of flavors, and/or different concentrations of other ingredients. The two or more ejector plates to which the two or more nicotine-containing formulations are supplied can have the same specifications or can be different from each other. The two or more ejector plates may differ in specification (e.g., hole size) to deliver different amounts of solution/aerosol to a user and/or to deliver different particle sizes to a user. The hole size of the ejector plates may be different in order to affect where the aerosol from each ejector plate/formulation combination deposits in the body (i.e., mouth, upper airway, throat, lungs, etc.).

In some embodiments, one nicotine-containing formulation is supplied to two or more ejector plates. The ejector plates may have different hole sizes and/or other specifications to affect the characteristics of the produced aerosols (i.e., particle size, mass ejection, etc.) and where they deposit in the body.

In some embodiments, one or more of the fluids delivered contains one or more therapeutic agents. Non-limiting examples include immunotherapeutic drugs, COPD medications, asthma medications, and the like. In some embodiments, one or more of the fluids contains one or more therapeutic agents to be delivered to specific areas of the respiratory system, and a second fluid contains a flavor to be delivered to the mouth and/or upper airway. This could make medications more palatable for users.

In some embodiments, formulations and devices can be used for Traditional Chinese Medicine (TCM). This could include but not limited to ginseng, ginsenosides, panax ginseng, ephedra, licorice root, Shuanghuanglian, Yuxingcao, Qingkailing, Ephedrae herba, Paeoniae radix, Glycyrrhizae radix, Cinnamonomi ramulus, Asari herba cum radice, bitter apricot seed, Common perilla stem, Ledebouriella root, Trichosanthes kirilowii Maxim (Gualoupi), Allium macrostemon Bunge, Puerariae Lobatae Radix, Salvia miltiorrhiza Bunge, Astragalus mongholicus Bunge, Davallia trichomanoides Blume, Paeonia lactiflora Pall, Alisma plantago-aquatica L., Ligusticum chuanxiong Hort, Curcuma aromatica Salisb, ling zhi (Ganoderma lucidum, commonly known as reishi), gan cao (Glycyrrhiza uralensis, commonly known as Chinese liquorice), ku shen (Sophora flavescens, commonly known as shrubby sophora, Mai Men Dong (Radix Ophiopogonis), Ban Xia (Rhizoma Pinelliae), American Ren Shen (Radix Panacis Quinquefolii), Lantern Tridax (Herba Tridacis procumbentis), Mai Men Dong (Radix Ophiopogonis), Ban Xia (Tuber Pinellia), Shu Di Huang (Radix Rehmanniae Preparata), Mu Dan Pi (Cortex Moutan Radicis), Shan Zhu Yu (Fructus Corni), Fu Ling (Poria), Ze Xie (Rhizoma Alismatis), Shan Yao (Radix Dioscoreae), Ma Huang (Herba Ephedrae), Xing Ren (Semen Armeniacae Amarum), Shi Gao (Gypsum Fibrosum), Gan Jiang (Rhizoma Zingiberis), Xi Xin (Herba Asari), Wu Wei Zi (Fructus Schisandrae), Bai Shao Yao (Radix Paeoniae), Bai Guo (Semen Ginkgo), Kuan Dong Hua (Flos Farfarae), Sang Bai Pi (Cortex Moris), Su Zi (Fructus Perillae), and Huang Qin (Radix Scutellariae).

Two or more fluids with TCM formulation(s) can be supplied to one or more ejector plates in a device. The fluids can contain the same TCM formulation or the formulations can be different. Various embodiments of fluids are listed below.

In some embodiments, compositions for TCM formulation herein may include water at a concentration of about 10% (w/w) to about 95% (w/w). In some embodiments, compositions for TCM formulation herein may include water at a concentration of 0% (w/w) to about 100% (w/w).

In some embodiments, compositions for TCM formulation herein may comprise of propylene glycol at a concentration of 0% (w/w) to about 20% (w/w). In some embodiments, compositions for TCM formulation herein may comprise of propylene glycol at a concentration of 0% (w/w) to about 100% (w/w).

In some embodiments, compositions for TCM formulation herein may comprise of ethanol at a concentration of 0% (w/w) to about 50% (w/w). In some embodiments, compositions for TCM formulation herein may comprise of ethanol at a concentration of 0% (w/w) to about 100% (w/w).

In some embodiments, compositions for TCM formulation herein may comprise of cyclodextrin at a concentration of 0% (w/w) to about 5% (w/w). In some embodiments, compositions for TCM formulation herein may comprise of cyclodextrin at a concentration greater than 5% (w/w). Cyclodextrin is used as a solubilizing agent.

In some embodiments, various combinations of water, propylene glycol, ethanol, and/or cyclodextrin can be used to solubilize the TCMs.

In various embodiments, combinations of any of the formulations outlined in the present disclosure may be supplied to one or more ejector plates in a droplet delivery device.

In various embodiments, combinations of any of the formulations outlined in the present disclosure, may be supplied to one or more ejector plates in a droplet delivery device. The ejector plates can have the same specifications (i.e., hole size) for delivery to the same regions of the respiratory system. Or, the ejector plates can have different specifications (i.e., hole size) to affect the aerosol characteristics (i.e., particle size) produced, which will cause the aerosols to be deposited in different regions of the respiratory system.

In some embodiments, two or more formulations may be supplied to one ejector plate, which is configured to eject the two or more formulations at different times. Alternatively, the ejector plate can be configured to eject the two or more formulations at the same time.

### Divider and Modulated Ejection

In one embodiment with more than one fluid and ejector plates, it is not desirable to have the fluid mix once the ejected streams of droplets become airborne. There is a possibility that droplets of one ejected stream will adhere to droplets of the second ejected stream. To mitigate this possibility, in some embodiments ejection from the ejector plates is time-multiplexed (i.e., the ejector plates are not actuated simultaneously) to reduce interaction between droplets from different streams. The ratio for time of ejection for each ejector plate can be 1:1, 1:2, 1:3, 1:4, 1:5, 1:6, 1:7, 1:8, 1:9, 1:10, 1:15, 1:20, 1:25, 1:30, 1:40, 1:50, 1:75, 1:100, and the like.

In the case of having more than one ejector plate where each one targets a different area of the respiratory system, it is likely that the smaller droplets will be actuated for a longer total time because the larger droplets will deliver a larger volume of liquid while ejecting. Therefore, the larger droplet ejector will not need to be on more than the smaller ejector.

Additionally, a barrier (112) can be in place to separate the streams of droplets. This will help prevent the two ejected streams of droplets from mixing in the mouthpiece (1011).

### Control

It may be desirable for the user to control the amount of ejection from each ejector plate, independently. In one embodiment, the device is configured to be programmed to allow the user to change the amount of ejection using the peripherals on the device. The peripherals can include, but are not limited to, a button (1014), accelerometer (shaking or tapping the device), capacitive sensor(s), and the like.

In another embodiment, the device is connected to a smartphone. The device can be connected to a smartphone via Bluetooth, near-field communication (NFC), or the like. The smartphone will have an app. This app can allow the user to control the amount of ejection from each ejector plate. The app can have, as a non-limiting example, sliders to control the amount of ejection. In another non-limiting example, the app can have preset values for the ejection volumes in selectable buttons.

In another embodiment, the device is programmed to control the amount of ejection for each ejector plate. This can be based upon a prescription. In a non-limiting example, the prescription may require the volume of agent delivered to decrease over time. The device will automatically decrease the ejection of this agent as directed. In another non-limiting example, the volume of agent delivered could need to increase over time. In this case, the device will automatically increase the ejection of the agent as directed. The taper or increase could be based upon a predetermined regimen set by a doctor or physician.

In one embodiment, this could be used with smoking cessation/nicotine cessation. The amount of nicotine delivered would decrease over time.

In another embodiment, the device is programmed to control the amount of ejection for each ejector plate. This can be based upon a prescription and could be used for the delivery of one or more steroid(s). The steroid needs to be delivered in a high volume at first and slowly tapers off as it is used. The taper could be based upon a predetermined regimen set by a doctor or physician.

In another embodiment, the device changes the amount of ejection for each of the ejector plates based upon the needs of the user. This could be based upon health data of the user, current air quality, or inhalation speed.

In another embodiment, the device is programmed to control the amount of ejection for each ejector plate. This can be based upon user preference. In a non-limiting example, when using a non-prescriptive device such as one that delivers a nicotine-containing formulation, the user may desire to decrease their consumption over time. The device will automatically decrease the ejection of this agent as directed. As another non-limiting example, a user may wish to increase the amount of flavor being delivered from a secondary ejector while they are decreasing their consumption of nicotine-containing formulation from the first ejector. They could independently direct the nicotine ejector to decrease delivery over time while increasing the delivery of the flavor ejector.

In some embodiments, the amount of ejection is controlled by power level or PWM. Changing the power level delivered to the piezoelectric transducer will also change the droplet size, or mass median aerodynamic diameter (MMAD). Therefore, the user and/or device can customize the ejection amount and/or the MMAD. MMAD means that 50% of particles in the aerodynamic size distribution, based on mass, lie above and below that diameter.

In yet another embodiment, the device will have one ejector plate to deliver an ejected stream of droplets to the lower airways and lungs. The device will have a second ejector plate to deliver an ejected stream of droplets to the upper airways, mouth, and throat.

In another embodiment, the user can choose different ejectors to purchase. The ejectors would be interchangeable in the device. Each ejector could have different characteristics, such as a different aperture size in the ejector plate. In this way, the user could customize the characteristics of the aerosol produced by the device (e.g., particle size, mass ejection, etc.). For example, a person using a nicotine delivery device may desire an aerosol with more irritation/throat hit. In this case, they may choose to buy ejector(s) with a larger aperture size that will deliver larger droplets. On the other hand, if a user desires a smoother aerosol with less irritation, they may choose to buy ejector(s) with a smaller aperture size that deliver smaller droplets.

Table 1 and Table 2 outline ejected mass and droplet size data of ejected streams of droplets with different aperture sizes. The droplet size was measured using two instruments, the Malvern Spraytec and the Dekati ELPI+. The Malvern Spraytec returns the droplet size as the d50. The d50 is the fifty-percentile mark in the droplet size distribution. The Dekati ELPI+ returns the droplet size as the MMAD.

Each Dekati ELPI+ pulls at a constant flow rate of around 10 LPM. This particular instrument is set to pull at a constant 9.81 LPM. The device is connected to a tube that goes to a tee. The tee connects to a compressed air inlet and the Dekati ELPI+. The compressed air inlet is connected to a mass flow controller set to 7.81 LPM. This means that the rest of the airflow must come through the device at 2 LPM. The tee fitting is special in that the aerosol inlet of the tee extends past the compressed air inlet and allows the compressed air inlet to provide sheath airflow. This minimizes any turbulence in the aerosol path. The aerosol then goes through a calibrated diluter at 100:1 before entering into the Dekati ELPI+.

The test method of the Dekati ELPI+ includes ejecting the device three times into the instrument. The instrument takes an average MMAD of the three ejections. This is done three times to get an average of the averages. Additionally, the mass of the whole device is measured before ejection and after each set of three ejections. The difference in mass is divided by three to get an average ejected mass. This is also done for each of the three sets of ejections to get an average of the average ejected mass.

The Malvern Spraytec is setup very similarly. The inhalation cell is attached to the Spraytec to enclose the system to ensure airflow through the device. The airflow is set at 10 LPM through the inhalation cell. The air purge on the inhalation cell is used to bring in ambient air and to split the airflow. This allows the device to be actuated with 2 LPM and the ambient air pulls in 8 LPM. The air purge also provides sheath air flow around the aerosol to minimize turbulence.

The test method of the Malvern Spraytec is nearly identical to the Dekati ELPI+. The device is ejected three times into the Spraytec. The software measures each individual d50. The average of the three d50s is taken. This is done three total times to get an average of averages. Additionally, the mass is measured before and after each set of three ejections to get three total ejected mass measurements. The average of these is taken to get an average of averages for the pierted mass

TABLE 1
The table below is ejection mass and droplet size data of ejected streams
of droplets generated from ejector plates with different aperture
sizes. This data was collected using an apple flavor solution.

| | | |
|---|---|---|
| Aperture | Malvern Spraytec | Dekati ELPI+ |

| size | Ejected mass | d50 | Ejected Mass | MMAD |
|---|---|---|---|---|
| (μm) | (mg) | (μm) | (mg) | (μm) |
| 2.5 | 3.19 | 2.47 | 3.61 | 1.19 |
| 3.0 | 5.8 | 3.26 | 7.19 | 2.42 |
| 3.5 | 6.51 | 4.15 | 8.02 | 2.85 |
| 3.9 | 7.18 | 4.65 | 9.62 | 3.15 |

TABLE 2
The table below is ejection mass and droplet size data of ejected streams
of droplets generated from ejector plates with different aperture
sizes. This data was collected using a cool mint flavor solution.

| | | |
|---|---|---|
| Aperture | Malvern Spraytec | Dekati ELPI+ |

| size | Ejected mass | d50 | Ejected Mass | MMAD |
|---|---|---|---|---|
| (μm) | (mg) | (μm) | (mg) | (μm) |
| 2.5 | 3.00 | 4.09 | 3.22 | 3.60 |
| 3.0 | 4.13 | 4.29 | 5.21 | 3.27 |
| 3.5 | 5.86 | 4.48 | 6.11 | 3.41 |
| 3.9 | 6.72 | 5.16 | 6.78 | 3.43 |

### Mouthpiece

The shape of the mouthpiece (1011) used with a device can affect the delivered droplets. A smaller droplet size aerosol can easily change direction inside the mouthpiece (1011). A larger droplet size aerosol will have more momentum and cannot make turns. Therefore, in one embodiment, the airflow exit (111) of the mouthpiece (1011) is directly in-line with the ejector plate for the larger aerosol. An example of this is shown in FIG. 10. This will ensure the larger droplets do not impinge on any wall inside the mouthpiece (1011).

### Safer

In some embodiments, the droplet delivery device does not rely on bulk heating of liquid to generate aerosol and can deliver primarily water-based droplets. In embodiments, reducing or avoiding heating may reduce formation of certain thermally generated byproducts relative to heated aerosol generation approaches. By separately delivering a nicotine-containing formulation and a flavor and/or sensate formulation to different regions of the respiratory tract, the device may reduce delivery of certain flavoring constituents to the lower airway while maintaining a desired sensory experience. In some embodiments, a nicotine salt formulation is primarily delivered to the lower airway/lungs, while flavoring is primarily delivered to the mouth and/or upper airway, such that a portion of the flavoring may be swallowed rather than inhaled into the lower airway.

### Nicotine Tapering/Smoking Cessation

In some embodiments, a device of the present application can be used for nicotine replacement therapy (NRT), smoking cessation, and/or nicotine cessation or tapering. In some embodiments, a nicotine-containing solution is provided to one ejector, and a flavor-containing solution is provided to a second ejector in a device. The ejectors can be controlled independently. The device can be programmed to taper the ejection of the nicotine-containing solution, while keeping the ejection of the flavor-containing solution constant. Alternatively, the ejection of the nicotine-containing solution can be tapered while the ejection of the flavor-containing solution is increased. In this way, the increase in flavor could help compensate for the decrease in nicotine in terms of user sensory experience. The taper in nicotine ejection could be programmed to be a linear taper over a certain amount of time (e.g., 3 weeks, 2 months, 4 months, etc.). The taper could be set to finish with the user receiving a fixed, lower amount of nicotine compared to when they began using the device. Alternatively, the taper in nicotine ejection could be set to finish with the user receiving no nicotine at the end of the taper period, while still receiving the flavor-containing solution. In a non-limiting example, at the end of a taper period, an ejector supplied with nicotine-containing solution, or originally supplied with nicotine-containing solution, could be programmed to not eject at all upon inhalation, while an ejector supplied with a flavor-containing solution is programmed to eject upon inhalation.

In some embodiments, a taper in nicotine ejection is pre-programmed to occur over a specified time period. The taper can be linear or can be any pre-determined pattern that may be more beneficial for a user. The taper time period and pattern can be based on a prescription or the recommendation of a doctor or physician.

The taper time period and pattern can be pre-programmed into a device by a pharmacy or medical office. Or, the device can be connected to an app or interface to allow the taper to be set remotely.

In some embodiments, a taper in nicotine ejection is dynamic and can be adjusted throughout the taper period. The taper time period and pattern can be determined by the user. The user can set the time period and pattern at the beginning of the taper period. This can be done by direct programming of the device or by connecting the device to an app or other interface. The taper time period and pattern can either be programmed to remain the same throughout the period or can be adjustable according to the needs of the user throughout their use. The user can also control the amount of flavor delivered throughout the nicotine tapering period.

In some embodiments, nicotine tapering can be done by altering the solution supplied to an ejector in a device. As part of a nicotine tapering regimen, the user may switch to solutions containing lower concentrations of nicotine at specified intervals. In this way, the device could continue to eject the same amount of solution throughout the tapering period, while delivering less nicotine. In a device with multiple ejectors, a flavor-containing solution supplied to one ejector could remain consistent throughout the nicotine tapering period, while the nicotine concentration of a nicotine-containing solution is gradually reduced. This regimen could end with the flavor-containing solution remaining consistent and the second solution no longer containing any nicotine. In some embodiments, the flavor concentration or ejection could increase while the concentration of nicotine is decreased throughout the regimen.

In some embodiments, a device of the present application is prescribed by a physician or doctor. In some embodiments, a device of the present application is available over-the-counter. In other embodiments, a device of the present application is available as a consumer device.

### Examples

Tables 4 and 5 contain particle size data collected from aerosol produced from various exemplary device embodiments. Particle testing was performed using the Dekati ELPI+ which is an electrical low-pressure impactor. The impactor is operated via the ELPI+VI software running on an external computer. The ELPI+ is connected to a Topaz model DIL 557 diluter with a ratio of 1:100. An external air tube and filter is attached to a flowrate device which is then connected by a T-connector. The other connection point is attached to a flexible tube that allows for the device to be connected. The combined airflow then goes into the diluter before entering the ELPI+. Since the ELPI+ is +/−0.2 LPM our EPLI+ flowrate was measured at 9.8 LPM. Due to this predetermined flowrate, the flowrate for the external air is set at 7.8 LPM, which means that the flowrate of the attached device would be equal to 2 LPM.

The data included in Tables 4 and 5 includes the mass median aerodynamic diameter (MMAD) of the aerosol, as well as d10 and d90 points. The d10 and d90 points are the droplet sizes at which 10% or 90% of the mass is less than that size, respectively.

TABLE 3
The table below outlines the formulations used to collect
the data shown in Table 4. The apple and tobacco flavorings
are manufactured by FLAVORIQ (Item Numbers: FQ.PNR0004
E-F Apple Sweet and FQ.PNR0009 E-F USA Mix).

| Solution | |
|---|---|
| Name | Formulation Composition |
| PB1 | 0.6% (w/w) nicotine lactate at pH of 5.2 in water |
| Tobacco | 0.6% (w/w) nicotine lactate at pH of 5.2, |
| | propylene glycol 5% (w/w), tobacco flavoring <0.05% |
| | (w/w), neotame 0.15% (w/w) in water |
| Apple | 0.6% (w/w) nicotine lactate at pH of 5.2, propylene |
| | glycol 10% (w/w), apple flavoring 0.3% (w/w), |
| | neotame 0.15% (w/w) in water |
| CM1 | 0.6% (w/w) nicotine lactate at pH of 5.2, propylene |
| | glycol 10%, ethanol 3% |
| CM2 | 0.6% (w/w) nicotine lactate at pH of 5.2, propylene |
| | glycol 10%, ethanol 10% |

As a non-limiting example, a droplet delivery device with 2 ejector plates in fluid communication with 2 separate fluid reservoirs, as shown in FIG. 13, was tested with various solutions. Each fluid reservoir was filled with the same solution for each part of the testing, so that the 2 ejector plates were supplied with the same solution. With each solution, the device was tested with both ejector plates programmed to actuate. The device was also tested with only one ejector plate programmed to actuate, which was done for both ejector plates. The data collected is shown in Table 4. The solutions used in the testing are outlined in Table 3.

TABLE 4
The table below shows the particle sizes of aerosol produced
by a droplet delivery device with 2 ejector plates in
fluid communication with various solutions. Each data
point shown in the table is the average of 3 actuations.

| Solution | Ejector Setup | MMAD (μm) | d90 (μm) |
|---|---|---|---|
| Apple 0.6% | Dual ejector | 0.529 | 1.124 |
| | Single 1 | 0.474 | 0.941 |
| | Single 2 | 0.533 | 1.123 |
| PB1 0.6% | Dual ejector | 0.551 | 1.183 |
| | Single 1 | 0.516 | 1.054 |
| | Single 2 | 0.536 | 1.145 |
| Tobacco 0.6% | Dual ejector | 0.531 | 1.146 |
| | Single 1 | 0.476 | 0.937 |
| | Single 2 | 0.517 | 1.095 |
| CM1 0.6% | Dual ejector | 0.490 | 1.030 |
| | Single 1 | 0.446 | 0.904 |
| | Single 2 | 0.494 | 1.035 |
| CM2 0.6% | Dual ejector | 0.430 | 0.871 |
| | Single 1 | 0.400 | 0.819 |
| | Single 2 | 0.420 | 0.857 |

As a non-limiting example, a droplet delivery device with 4 ejector plates in fluid communication with 4 separate fluid reservoirs was tested. Each fluid reservoir was filled with the same solution for each part of the testing, so that the 4 ejector plates were all supplied with the same solution. The device was tested with 4 concentrations of nicotine lactate (0.45% (w/w), 0.6% (w/w), 0.9% (w/w), and 1.8% (w/w)) at a pH of 5.2 in water. The results of the testing are shown in Table 5.

TABLE 5
The table below contains the particle sizes of aerosol produced
by a droplet delivery device with 4 ejector plates when supplied
with various concentrations of nicotine lactate.

| | | d10 | MMAD | d90 | |
|---|---|---|---|---|---|
| Solution | Actuation number | (μm) | (μm) | (μm) | GSD |
| 0.45% (w/w) | 1 | 0.197 | 0.469 | 0.969 | 1.813 |
| nicotine | 2 | 0.201 | 0.474 | 0.946 | 1.788 |
| lactate | 3 | 0.210 | 0.483 | 0.941 | 1.753 |
| | Average | 0.203 | 0.475 | 0.952 | 1.784 |
| | Standard Deviation | 0.006 | 0.007 | 0.015 | 0.030 |
| | CV (%) | 3.162 | 1.503 | 1.551 | 1.689 |
| 0.6% (w/w) | 1 | 0.336 | 0.708 | 1.475 | 1.835 |
| nicotine | 2 | 0.336 | 0.706 | 1.467 | 1.832 |
| lactate | 3 | 0.334 | 0.698 | 1.444 | 1.825 |
| | Average | 0.336 | 0.704 | 1.462 | 1.830 |
| | Standard Deviation | 0.001 | 0.006 | 0.016 | 0.005 |
| | CV (%) | 0.323 | 0.801 | 1.123 | 0.280 |
| 0.9% (w/w) | 1 | 0.304 | 0.639 | 1.342 | 1.815 |
| nicotine | 2 | 0.322 | 0.654 | 1.358 | 1.808 |
| lactate | 3 | 0.328 | 0.663 | 1.369 | 1.799 |
| | Average | 0.318 | 0.652 | 1.356 | 1.807 |
| | Standard Deviation | 0.012 | 0.012 | 0.013 | 0.008 |
| | CV (%) | 3.859 | 1.894 | 0.991 | 0.424 |
| 1.8% (w/w) | 1 | 0.378 | 0.805 | 1.555 | 1.750 |
| nicotine | 2 | 0.385 | 0.820 | 1.574 | 1.741 |
| lactate | 3 | 0.391 | 0.832 | 1.589 | 1.736 |
| | Average | 0.385 | 0.819 | 1.573 | 1.742 |
| | Standard Deviation | 0.007 | 0.014 | 0.017 | 0.007 |
| | CV (%) | 0.715 | 0.659 | 0.986 | 1.243 |

In some embodiments, multiple ejectors are included in a device for redundancy. This redundancy could present a particular advantage when a device is used to deliver critical drugs. For example, if a device delivers a medication that is critical to a user's health, having multiple ejectors could ensure the drug continues to be delivered even if one ejector fails. If one ejector fails, the device could switch to a back-up ejector. The back-up ejector could be programmed to not eject unless the primary ejector fails. Additionally, if one ejector fails, the device could compensate by delivering more out of the remaining ejector(s).

In some embodiments, a device can include one or more heated ejection systems and one or more non-heated ejection systems. This may be advantageous if it is necessary or preferred for one liquid to be aerosolized using a heated system, while another liquid is aerosolized using a non-heated system. The inclusion of one or more heated ejection systems may also be used to change the characteristics of one liquid or aerosol without affecting the other liquid or aerosol. For example, heating a liquid will change its surface tension and viscosity, which may be advantageous in some scenarios. As another example, using heat to generate an aerosol may be advantageous for the sensory experience when delivering some formulations. Another example, using heat can mitigate freezing of the liquid or liquids.

In some embodiments, multiple ejectors are included in a device to help blend, mix, or beat the liquid being delivered. One or more of the ejector plates may not have holes and may serve only to mix the liquid, rather than aerosolize it. This may be advantageous when delivering a suspension, non-homogeneous, or near immiscible liquid that needs to be mixed to a homogenous state immediately before aerosolization. In some embodiments, the mixing could take place in a mixing chamber that contains the one or more ejector plates intended to mix the liquid. The mixing chamber could be connected to one or more ejector plates meant to aerosolize the liquid. The device could be designed such that after mixing, the liquid is dispensed to or allowed to flow freely to the aerosolization ejector plates.

In some embodiments, two or more ejectors are included in a droplet delivery device. The device is designed such that one ejector ejects onto the aerosol plume of a second ejector at an angle. This can affect the flow of the droplets. One of the ejectors can have bigger droplets than the other so the flow of the droplets can be more or less manipulated. Additionally, when two aerosol paths collide, they may adhere to each other. This may also affect other aspects of the aerosol delivery.

In some embodiments, two or more ejectors are included in a droplet delivery device. One ejector may be programmed to eject first to deposit a layer or coating on the targeted location in the respiratory pathway. This layer may make the location have better bioavailability. A second ejector may be programmed to eject after the first ejector to deliver the necessary dose of liquid onto the coated section of the respiratory pathway. The layer created by the first ejector may enhance the absorption of the aerosol delivered by the second ejector.

In some embodiments, two or more ejectors are included in a droplet delivery device. One or more ejectors are used to deliver a substance that may be caustic or irritating to the user, while one or more ejectors are used to deliver a numbing agent or sensate to make the irritating substance more tolerable to the user. The numbing agent or sensate may be delivered prior to the irritating substance or at the same time as the irritating substance.

In some embodiments, two or more ejectors are included in a droplet delivery device. One or more ejectors may be used to deliver a substance that may be caustic or irritating. The droplet of the aerosol delivered may be selected to intentionally create irritation in one or more locations in the respiratory pathway of the user. For example, someone using a nicotine delivery device may desire some irritation in the throat or lungs as part of the sensory experience.

TABLE 6
The table below lists the features and/or components of the device.

| Element | |
|---|---|
| Number | Description |
| 11 | Upper airway aerosol |
| 12 | Lower airway aerosol |
| 111 | Airflow exit |
| 112 | Barrier |
| 113 | Upper airway ejector plate |
| 114 | Lower airway ejector plate |
| 115 | Vibrating member |
| 116 | Membrane |
| 411 | Open volume |
| 511 | Rod |
| 911 | Vibrating member with multiple extrusions |
| 1011 | Mouthpiece |
| 1012 | Cartridge |
| 1013 | Handpiece |
| 1014 | Button |
| 1015 | Battery |
| 1016 | PCBA |
| 1017 | Ejector bracket |
| 1018 | LED lights |

While the invention is disclosed in exemplary embodiments, it is not intended to be limited to the specific examples disclosed herein but extends to the full extent of the disclosure.

What is claimed is:

1. A droplet delivery device for delivery of more than one fluid to the respiratory system of a user, the more than one fluid delivered as one or more ejected stream of droplets, the droplet delivery device comprising:
a housing including a mouthpiece located at an airflow exit of the housing;
multiple fluid reservoirs disposed within or in fluid communication with the housing including a first fluid reservoir and a second fluid reservoir; and
at least one piezoelectric vibrating member operably coupled to a plurality of ejector plates, wherein each plate has a plurality of openings and at least one membrane between the at least one vibrating member and the plurality of ejector plates.

2. The droplet delivery device of claim 1, wherein the first fluid reservoir supplies a fluid including nicotine and the second fluid reservoir supplies a fluid including flavoring.

3. A method comprising ejecting droplets from the droplet delivery device of claim 2 and causing delivery of nicotine to the lungs having a respirable fraction of at least about 90%.

4. A method comprising ejecting droplets from the droplet delivery device of claim 2 and causing delivery of flavoring to the upper airway having a respirable fraction of at most about 30%.

5. The droplet delivery device of claim 2, wherein a first ejector plate of the plurality of ejector plates ejects nicotine from openings of the first ejector plate having an average aperture diameter of about 1 μm to 3 μm.

6. The droplet delivery device of claim 5, wherein a second ejector plate of the plurality of ejector plates ejects flavoring from openings of the second ejector plate having an average aperture diameter of about 2 μm to 30 μm.

7. The droplet delivery device of claim 2, wherein the mouthpiece has a barrier separating nicotine aerosol and flavor aerosol.

8. The droplet delivery device of claim 1, wherein the multiple reservoirs, the multiple ejector plates, the vibrating member and the at least one membrane are all contained within the housing.

9. The droplet delivery device of claim 1, wherein the multiple reservoirs, the multiple ejector plates, the at least one vibrating member, the at least one membrane and the housing are all in a linear arrangement along a common axis.

10. The droplet delivery device of claim 1 further comprising a plurality of piezoelectric vibrating members.

11. The droplet delivery device of claim 1, wherein the at least one vibrating member is a single piezoelectric vibrating member having a first vibrating tip coupled to a first membrane and a second vibrating tip coupled to a second membrane.

12. The droplet delivery device of claim 1 further comprising two or more membranes and wherein each membrane of said two or more membranes is coupled to a respective separate ejector plate of the plurality of ejector plates.

13. A droplet delivery device for delivery of more than one fluid to the respiratory system of a user, the more than one fluid delivered as one or more ejected stream of droplets, the droplet delivery device comprising:
a housing comprising a mouthpiece located at an airflow exit of the housing;
multiple fluid reservoirs disposed within or in fluid communication with the housing comprising a first fluid reservoir and a second fluid reservoir;
at least one piezoelectric vibrating member operably coupled to a plurality of ejector plates, wherein each plate has a plurality of openings and at least one membrane between the vibrating member and the plurality of ejector plates; and
a modulator configured to avoid continuously ejecting a first fluid of the more than one fluid from the multiple fluid reservoirs for the entirety of the inhalation and ejecting the first fluid in pulses.

14. The droplet delivery device of claim 13, wherein the first fluid reservoir contains a fluid with nicotine and the second fluid reservoir contains a fluid with flavoring.

15. A method comprising ejecting droplets from the droplet delivery device of claim 14 and causing delivery of nicotine to the lungs having a respirable fraction of at least about 90%.

16. A method comprising ejecting droplets from the droplet delivery device of claim 14 and causing delivery of flavoring to the upper airway having a respirable fraction of at most about 30%.

17. The droplet delivery device of claim 14, wherein a first ejector plate of the plurality of ejector plates ejects nicotine from openings of the first ejector plate having an average aperture diameter of about 1 μm to 3 μm.

18. The droplet delivery device of claim 14, wherein a second ejector plate of the plurality of ejector plates ejects flavoring from openings of the second ejector plate having an average aperture diameter of about 2 μm to 30 μm.

19. The droplet delivery device of claim 14, wherein the mouthpiece has a barrier separating nicotine aerosol and flavor aerosol.

20. The droplet delivery device of claim 13, wherein the multiple reservoirs, the multiple ejector plates, the vibrating member and the at least one membrane are all contained within the housing.

21. The droplet delivery device of claim 13, wherein the multiple reservoirs, the multiple ejector plates, the at least one vibrating member, the at least one membrane and the housing are all in a linear arrangement along a common axis.

22. The droplet delivery device of claim 13 further comprising a plurality of piezoelectric vibrating members.

23. The droplet delivery device of claim 13 wherein the at least one vibrating member is a single piezoelectric vibrating member having a first vibrating tip coupled to a first membrane and a second vibrating tip coupled to a second membrane.

24. The droplet delivery device of claim 13 further comprising two or more membranes and wherein each membrane of said two or more membranes is coupled to a respective separate ejector plate of the plurality of ejector plates.

25. The droplet delivery device of claim 13, wherein the housing has an elongated body defining a single longitudinal axis.

26. The droplet delivery device of claim 1, wherein the housing has an elongated body defining a single longitudinal axis.
